(19) United States
(12) Patent Application Publication
(10) Pub. No.: US 2023/0274790 A1
(43) Pub. Date: Aug. 31, 2023

(54) SYSTEMS, METHODS, AND MEDIA FOR CLASSIFYING GENETIC SEQUENCING RESULTS BASED ON PATHOGEN-SPECIFIC ADAPTIVE THRESHOLDS

(71) Applicant: Arc Bio, LLC, Cambridge, MA (US)

(72) Inventors: Alejandro QUIROZ-ZARATE, Cambridge, MA (US); Matt JOBIN, Santa Cruz, CA (US)

(21) Appl. No.: 18/008,004

(22) PCT Filed: Jun. 3, 2021
(86) PCT No.: PCT/US2021/035731

§ 371 Date: Dec. 2, 2022

Related U.S. Application Data
(60) Provisional application No. 63/034,332, filed on Jun. 3, 2020.

(51) Int. Cl. G16B 20/00; G16B 40/20; G16B 30/10; G06N 3/02

(52) U.S. Cl. CPC G16B 20/00; G16B 40/20; G16B 30/10; G06N 3/02

(57) ABSTRACT

In accordance with some embodiments, systems, methods, and media for classifying genetic sequencing results based on pathogen-specific adaptive thresholds are provided. In some embodiments, a system comprises a processor programmed to: receive negative control results, each comprising values indicative of a number of reads detected in the respective negative control sample for an organism; generate a model based on the negative control results; receive a clinical sample result for a clinical sample, comprising values indicative of a number of reads detected in the clinical sample for an organism of a plurality of organisms; identify, utilizing the model, any values in the clinical sample that are likely to be diagnostically significant; generate a report based on the clinical sample result and organisms associated with a value likely to be diagnostically significant; and cause the report to be presented to a user.

### CROSS-REFERENCE TO RELATED APPLICATIONS

This application is based on, claims the benefit of, and claims priority to U.S. Provisional Application No. 63/034,332, filed Jun. 3, 2020, which is hereby incorporated herein by reference in its entirety for all purposes.

### STATEMENT REGARDING FEDERALLY SPONSORED RESEARCH

N/A

### BACKGROUND

Genetic sequencing can identify genetic material present in a sample. However, even if the source of the genetic material is perfectly identified (e.g., assuming perfect identification of the organisms from which the material originated), there are many sources of contamination that may cause relatively small amounts of genetic material to show up in results that do not represent diagnostically relevant information about organisms present in the intended sample. For example, genetic material from a host can be identified which is often not relevant because the host is known, and the goal is to identify unknown organisms (e.g., pathogens) present in the host. As another example, genetic material from a diagnostically irrelevant organism can be identified, which may be considered a contaminant. In a more particular example, if a blood sample is being analyzed, organisms present on the skin of the host may have been inadvertently included in the sample. As yet another example, physical contamination in a laboratory (which can stem from staff at the laboratory, equipment, and/or reagents), may also come into contact with a sample prior to sequencing and thus possibly appear in the output. Additionally, genetic material can be misidentified as belonging to an organism of interest erroneously. For example, benign genetic material can be mistakenly included in a reference sequence for a pathogen. As another example, an alignment system can associate a read with an incorrect organism.

Another potential source of false positives is convergence and/or homoplasy, in which different organisms have portions of genetic sequences that match, even though the organisms are not closely related and the genetic sequence was not present in their common ancestor. A related source of false positives is symplesiomorphies in which certain genetic material was present in a common ancestor and is very widely shared by many species. Such genetic material can be misattributed to an organism that is not present in the sample if it is not filtered out.

These sources of error can lead to results being reported indicating the presence of many organisms that are unlikely to be present in a sample. However, because different organisms are diagnostically relevant at different concentrations, while various sources of error can lead to many false positive readings, low level results cannot be categorically ignored. Therefore, setting a single threshold across all organisms for determining relevance is undesirable.

Accordingly, new systems, methods, and media for classifying genetic sequencing results based on pathogen-specific adaptive thresholds are desirable.

### SUMMARY

In accordance with some embodiments of the disclosed subject matter, systems, methods, and media for classifying genetic sequencing results based on pathogen-specific adaptive thresholds are provided.

In accordance with some embodiments of the disclosed subject matter, a system for classifying a genetic sequencing result for a sample is provided, the system comprising: at least one hardware processor that is programmed to: receive a plurality of negative control sample genetic sequencing results corresponding to a respective plurality of negative control samples, each negative control sample genetic sequencing result comprises a plurality of values that are each indicative of a number of reads detected in the respective negative control sample for a respective organism of a plurality of organisms; generate a model based on the plurality of negative control sample genetic sequencing results; receive a clinical sample genetic sequencing result for a clinical sample, the clinical sample genetic sequencing result comprising a plurality of values that are each indicative of a number of reads detected in the clinical sample for a respective organism of the plurality of organisms; identify, utilizing the model, any values in the clinical sample genetic sequencing result that are likely to be diagnostically significant; generate a report based on the clinical sample genetic sequencing result and any organisms associated with a value identified as likely to be diagnostically significant; and cause at least a portion of the report to be presented to a user with an indication of any organisms associated with a value identified as likely to be diagnostically significant.

In some embodiments, the at least one hardware processor is further programmed to: generate a distribution for each of the plurality of organisms based on the plurality of negative control sample genetic sequencing results; associate, for each of the plurality of organisms, a threshold that is based on the distribution; and identify, utilizing the model, any values in the clinical sample genetic sequencing result that are likely to be diagnostically significant based on the threshold associated with the organism.

In some embodiments, the at least one hardware processor is further programmed to set the threshold for each of the plurality of organisms at the median of the distribution associated with that organism.

In some embodiments, the at least one hardware processor is further programmed to: train a neural network using the plurality of negative control sample genetic sequencing results; provide the clinical sample genetic sequencing result as input to the trained neural network; and receive, from the trained neural network, output identifying any values in the clinical sample genetic sequencing result that are likely to be diagnostically significant.

In some embodiments, the neural network is an autoencoder comprising: an input layer, the input layer comprising a plurality of nodes corresponding to respective organisms of the plurality of organisms; at least one encoding layer comprising no more than 15% of the number of nodes in the input layer; a coding layer comprising no more than 6.5% of the number of nodes in the input layer; at least one decoding layer comprising the same number of nodes as the at least one encoding layer; and an output layer, the output layer comprising a plurality of nodes corresponding to respective organisms of the plurality of organisms.

In some embodiments, the encoding layers comprises no more than 0% of the number of nodes in the input layer, and the coding layer comprises no more than 0.2% of the number of nodes in the input layer.

In some embodiments, the at least one hardware processor is further programmed to: receive a plurality of positive control sample genetic sequencing results corresponding to a respective plurality of positive control samples, each positive control sample genetic sequencing result comprises a plurality of values that are each indicative of a number of reads detected in the respective positive control sample for a respective organism of a second plurality of organisms; and train the autoencoder using the plurality of negative control sample genetic sequencing results and the plurality of positive control sample genetic sequencing results.

In some embodiments, the at least one hardware processor is further programmed to: generate a heatmap indicative of the values in the clinical sample genetic sequencing result; augment which the organisms are presented in the heatmap based on any organisms associated with a value identified as likely to be diagnostically significant; and cause at least a portion of the heatmap corresponding to the organisms associated with a value identified as likely to be diagnostically significant to be presented within the portion of the report.

In accordance with some embodiments, a method for classifying a genetic sequencing result for a sample is provided, the method comprising: receiving a plurality of negative control sample genetic sequencing results corresponding to a respective plurality of negative control samples, each negative control sample genetic sequencing result comprises a plurality of values that are each indicative of a number of reads detected in the respective negative control sample for a respective organism of a plurality of organisms; generating a model based on the plurality of negative control sample genetic sequencing results; receiving a clinical sample genetic sequencing result for a clinical sample, the clinical sample genetic sequencing result comprising a plurality of values that are each indicative of a number of reads detected in the clinical sample for a respective organism of the plurality of organisms; identifying, utilizing the model, any values in the clinical sample genetic sequencing result that are likely to be diagnostically significant; generating a report based on the clinical sample genetic sequencing result and any organisms associated with a value identified as likely to be diagnostically significant; and causing at least a portion of the report to be presented to a user with an indication of any organisms associated with a value identified as likely to be diagnostically significant.

In accordance with some embodiments of the disclosed subject matter, a non-transitory computer readable medium containing computer executable instructions that, when executed by a processor, cause the processor to perform a method for classifying a genetic sequencing result for a sample is provided, the method comprising: receiving a plurality of negative control sample genetic sequencing results corresponding to a respective plurality of negative control samples, each negative control sample genetic sequencing result comprises a plurality of values that are each indicative of a number of reads detected in the respective negative control sample for a respective organism of a plurality of organisms; generating a model based on the plurality of negative control sample genetic sequencing results; receiving a clinical sample genetic sequencing result for a clinical sample, the clinical sample genetic sequencing result comprising a plurality of values that are each indicative of a number of reads detected in the clinical sample for a respective organism of the plurality of organisms; identifying, utilizing the model, any values in the clinical sample genetic sequencing result that are likely to be diagnostically significant; generating a report based on the clinical sample genetic sequencing result and any organisms associated with a value identified as likely to be diagnostically significant; and causing at least a portion of the report to be presented to a user with an indication of any organisms associated with a value identified as likely to be diagnostically significant.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows an example of a system for classifying genetic sequencing results based on pathogen-specific adaptive thresholds in accordance with some embodiments of the disclosed subject matter.

FIG. 2 shows an example of hardware that can be used to implement a computing device, and a server, shown in FIG. 1 in accordance with some embodiments of the disclosed subject matter.

FIG. 3 shows an example of a process for determining pathogen-specific adaptive thresholds in accordance with some embodiments of the disclosed subject matter.

FIG. 4 shows an example of a process for generating a statistical model using negative control samples for determining pathogen-specific adaptive thresholds in accordance with some embodiments of the disclosed subject matter.

FIG. 5 shows an example of a process for generating a machine learning model using negative control samples or negative control samples and positive control samples for determining pathogen-specific adaptive thresholds in accordance with some embodiments of the disclosed subject matter.

FIG. 6 shows an example of a topology of an autoencoder that can be generated to predict pathogen-specific adaptive thresholds using mechanisms described herein in accordance with some embodiments of the disclosed subject matter.

FIG. 7 shows an example of a heatmap presenting sequencing and alignment results for various pathogens from a variety of clinical samples in in accordance with some embodiments of the disclosed subject matter.

FIG. 8A shows an example of a heatmap presenting sequencing and alignment results for various pathogens from a variety of positive control samples used to test models generated in accordance with some embodiments of the disclosed subject matter.

FIGS. 8B1 and 8B2 show examples of heatmaps presenting sequencing and alignment results for various pathogens from a variety of negative control samples used to test models generated in accordance with some embodiments of the disclosed subject matter.

FIG. 9 shows an example of a table presenting classification results for the variety of simulated samples generated by various models in accordance with some embodiments of the disclosed subject matter.

FIG. 10 shows an example of a table presenting classification results for a variety of samples generated by various models trained for different lengths of time in accordance with some embodiments of the disclosed subject matter.

FIG. 11 shows an example of a table presenting classification results for a variety of samples generated by various models using different decision rules in accordance with some embodiments of the disclosed subject matter.

FIG. 12A shows an example of a graph of sensitivity of various techniques used to classify a result as signal or noise in accordance with some embodiments of the disclosed subject matter.

FIG. 12B shows an example of a graph of specificity of various techniques used to classify a result as signal or noise in accordance with some embodiments of the disclosed subject matter.

FIG. 12C shows an example of a graph of precision of various techniques used to classify a result as signal or noise in accordance with some embodiments of the disclosed subject matter.

FIG. 13A shows an example of a graph of sensitivity of various techniques used to classify a result as signal or noise at different titrations in accordance with some embodiments of the disclosed subject matter.

FIG. 13B shows an example of a graph of specificity of various techniques used to classify a result as signal or noise at different titrations in accordance with some embodiments of the disclosed subject matter.

FIG. 13C shows an example of a graph of precision of various techniques used to classify a result as signal or noise at different titrations in accordance with some embodiments of the disclosed subject matter.

### DETAILED DESCRIPTION

In accordance with various embodiments, mechanisms (which can, for example, include systems, methods, and media) for classifying genetic sequencing results based on pathogen-specific adaptive thresholds are provided.

In accordance with some embodiments of the disclosed subject matter, mechanisms described herein can be used to generate a model that can classify results of genetic sequencing as more or less likely to be clinically significant. In general, a sample (e.g., blood, sputum, fecal matter, etc.) can be sequenced to attempt to identify organisms present in the sample. Next generation sequencing techniques can be used to relatively inexpensively and relatively quickly identify reads (e.g., on the order of dozens to thousands of base pairs in length) present in the sample. The reads can then be aligned to reference sequences for various organisms to attempt to identify which organism a particular read originated from.

Various sources of error can cause false positive results to be included in the aligned reads. One such source of error is low levels of pathogens present in a laboratory's environmental, which can lead to low level contamination of the sample, reagents, and/or equipment used to perform sequencing. The genetic material of the organisms present in the laboratory can be referred to as a labome of the laboratory. In general, each laboratory may have a different labome, which may be based on various factors such as the average microbiome of the population for which samples are analyzed, the microbiome of staff at the laboratory, the ambient environment, etc.

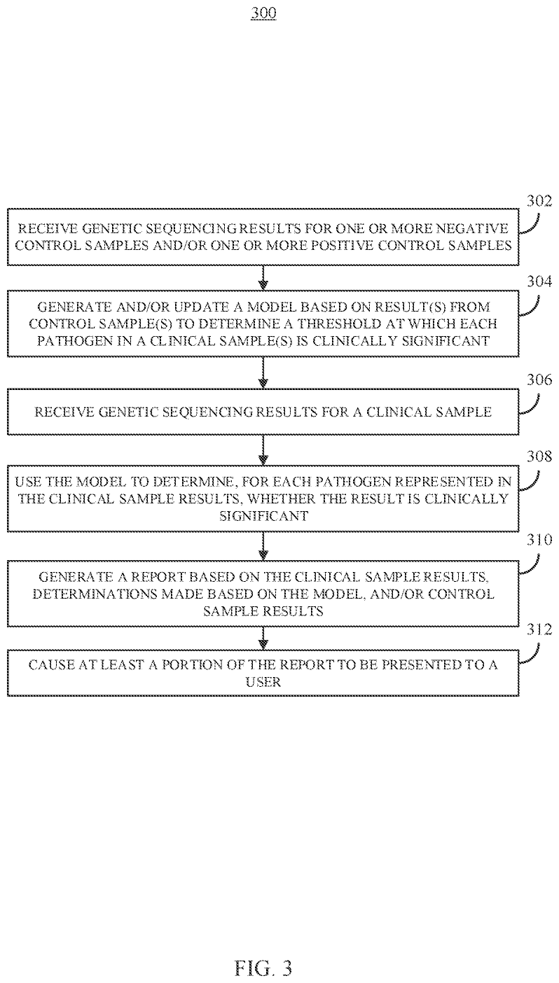

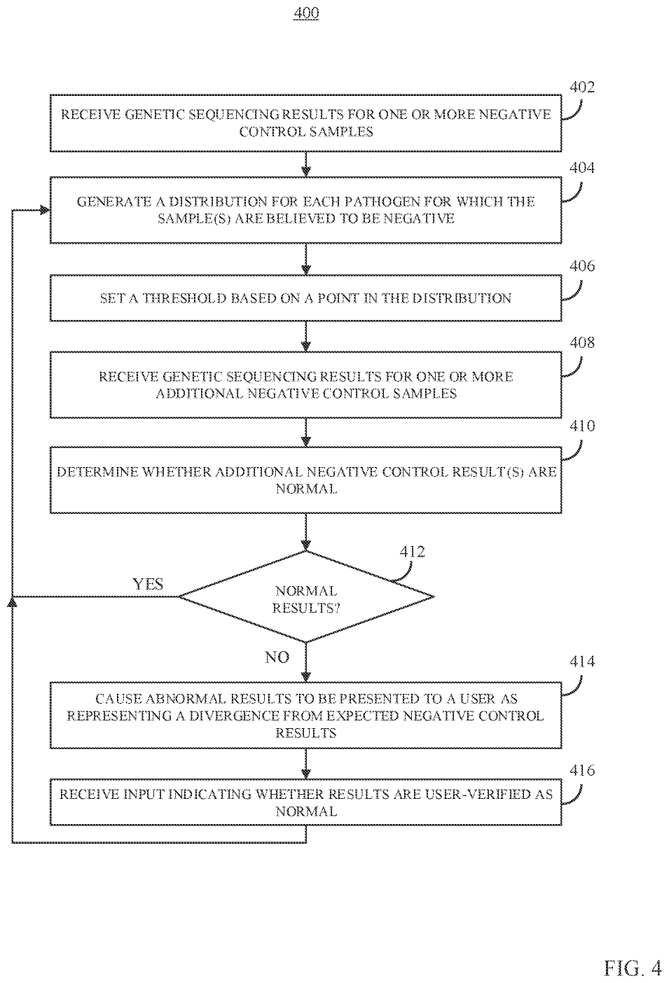

In some embodiments, mechanisms described herein can utilize information about the labome to determine whether a result for a particular organism in a sample is likely to be diagnostically or clinically significant. For example, as described below in connection with FIG. 3, results of negative control samples run at the laboratory can be used to generate a baseline model that can be used to determine whether a particular result is likely to be diagnostically significant. If the result exceeds the baseline present in the labome for that particular organism it can be indication that the organism was truly present in the sample, while if the result falls below the baseline present in the labome for that particular organism it can be an indication that the organisms is likely not present in the sample.

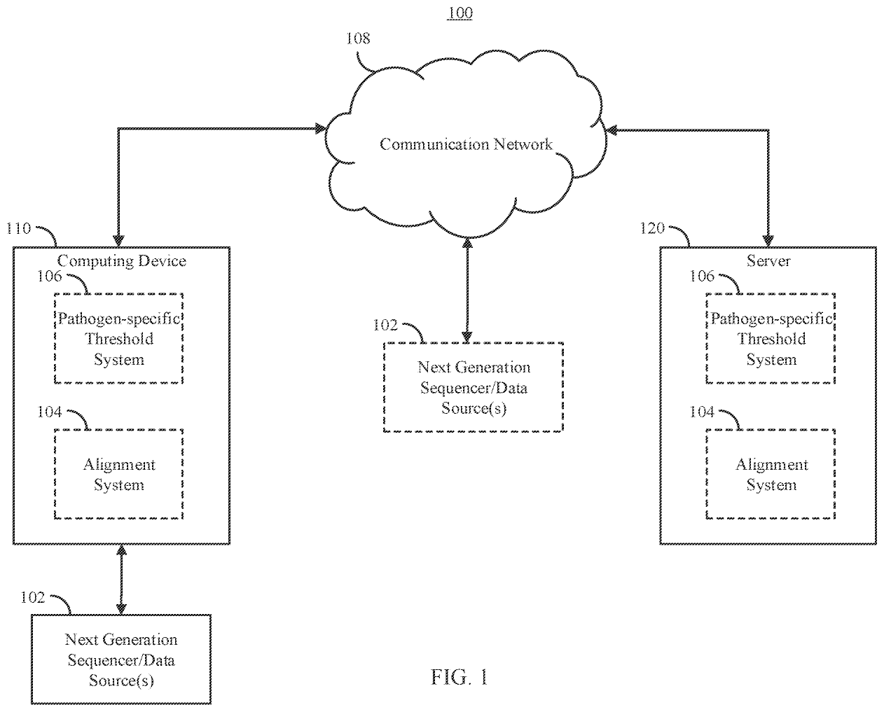

FIG. 1 shows an example of a system for classifying genetic sequencing results based on pathogen-specific adaptive thresholds in accordance with some embodiments of the disclosed subject matter. As shown in FIG. 1, a computing device 110 can receive sequencing results indicating genetic information (e.g., DNA, RNA, etc.) that is present in a sample (e.g., a clinical sample, a negative control sample, a positive control sample) from a data source 102 that generated and/or stores such data, and/or from an input device. In some embodiments, computing device 110 can execute at least a portion of an alignment system 104, and/or a pathogen-specific threshold system 106. In some embodiments, alignment system 104 can identify a correspondence between a read generated by a next generation sequencing device and a particular reference sequence (e.g., associated with a pathogen, associated with a likely source of contamination, etc.). In some embodiments, alignment system 104 can use any suitable alignment technique or combination of techniques, such as linear alignment techniques, and graph-based alignment techniques (e.g., as described in U.S. Patent Application Publication No. 2020/0090786, which is hereby incorporated by reference herein in its entirety).

In some embodiments, pathogen-specific threshold system 106 can generate a model (e.g., based on one or more negative control samples and/or positive control samples) that can be used to classify results associated with a particular pathogen as being consistent with negative controls (e.g., as being below a threshold), or as being indicative of presence of the pathogen in the sample being analyzed. For example, pathogen-specific threshold system 106 can execute one or more portions of processes 300, 400, and/or 500 described below in connection with FIGS. 3-5.

Additionally or alternatively, in some embodiments, computing device 110 can communicate information about genetic information (e.g., genetic sequence results generated by a next generation sequencing device, aligned reads associated with a particular reference sequence) from data source 102 to a server 120 over a communication network 108 and/or server 120 can receive genetic information from data source 102 (e.g., directly and/or using communication network 108), which can execute at least a portion of alignment system 104, and/or a pathogen-specific threshold system 106. In such embodiments, server 120 can return analysis results to computing device 110 (and/or any other suitable computing device) indicative of levels of one or more pathogens detected in a sample and/or a likelihood that the pathogen is a true positive in the sample.

In some embodiments, computing device 110 and/or server 120 can be any suitable computing device or combination of devices, such as a desktop computer, a laptop computer, a smartphone, a tablet computer, a wearable computer, a server computer, a virtual machine being executed by a physical computing device, a specialty device (e.g., a next generation sequencing device), etc. As described below in connection with FIGS. 3-5, in some embodiments, computing device 110 and/or server 120 can receive genetic data (e.g., corresponding to a positive control sample, a negative control sample, or a clinical sample) from one or more data sources (e.g., data source 102), and can associate portions of the genetic data with one or more reference genomes (e.g., using alignment system 104), and can generate a model that that can be used to classify results associated with a particular pathogen and/or use the model to classify results associated with a particular pathogen using pathogen-specific threshold system 106.

In some embodiments, data source 102 can be any suitable source or sources of genetic data. For example, data source 102 can be a next generation sequencing device or devices that generate a large number of reads from a sample. As another example, data source 102 can be a data store configured to store genetic data, which may be aligned genetic data or unaligned reads.

In some embodiments, data source 102 can be local to computing device 110. For example, data source 102 can be incorporated with computing device 110. As another example, data source 102 can be connected to computing device 110 by one or more cables, a direct wireless link, etc. Additionally or alternatively, in some embodiments, data source 102 can be located locally and/or remotely from computing device 110, and provide data to computing device 110 (and/or server 120) via a communication network (e.g., communication network 108).

In some embodiments, communication network 108 can be any suitable communication network or combination of communication networks. For example, communication network 108 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, WiMAX, 5G NR, etc.), a wired network, etc. In some embodiments, communication network 108 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

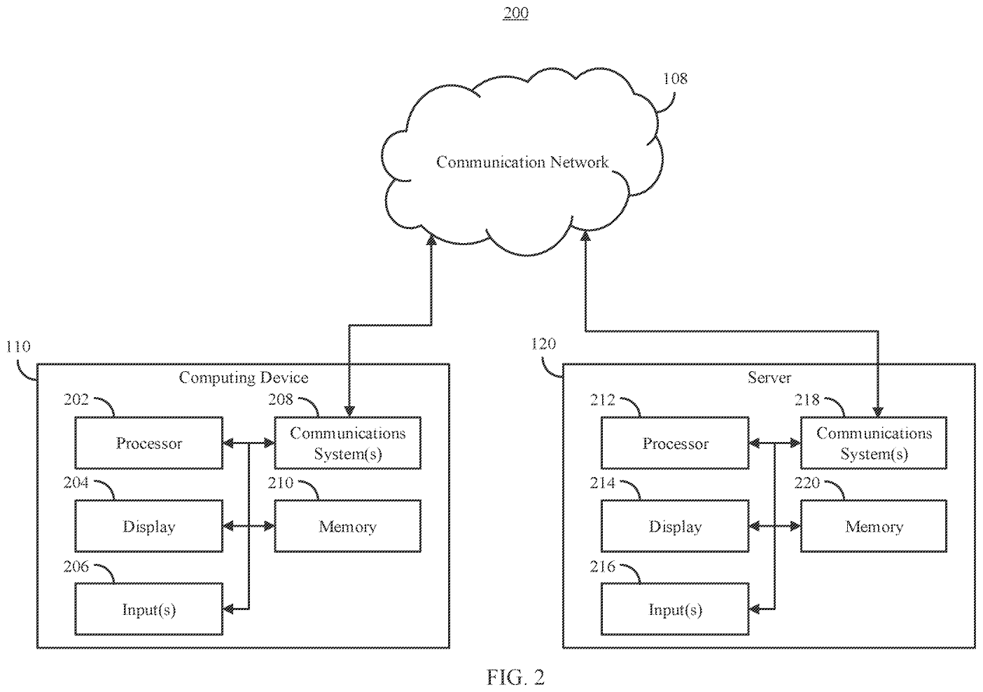

FIG. 2 shows an example 200 of hardware that can be used to implement computing device 110, and/or server 120 in accordance with some embodiments of the disclosed subject matter. As shown in FIG. 2, in some embodiments, computing device 110 can include a processor 202, a display 204, one or more inputs 206, one or more communication systems 208, and/or memory 210. In some embodiments, processor 202 can be any suitable hardware processor or combination of processors, such as a central processing unit (CPU), a graphics processing unit (GPU), a microcontroller (MCU), an application specification integrated circuit (ASIC), a field programmable gate array (FPGA), etc. In some embodiments, display 204 can include any suitable display devices, such as a computer monitor, a touchscreen, a television, etc. In some embodiments, inputs 206 can include any suitable input devices and/or sensors that can be used to receive user input, such as a keyboard, a mouse, a touchscreen, a microphone, etc.

In some embodiments, communications systems 208 can include any suitable hardware, firmware, and/or software for communicating information over communication network 108 and/or any other suitable communication networks. For example, communications systems 208 can include one or more transceivers, one or more communication chips and/or chip sets, etc. In a more particular example, communications systems 208 can include hardware, firmware and/or software that can be used to establish a Wi-Fi connection, a Bluetooth connection, a cellular connection, an Ethernet connection, etc.

In some embodiments, memory 210 can include any suitable storage device or devices that can be used to store instructions, values, etc., that can be used, for example, by processor 202 to present content using display 204, to communicate with server 120 via communications system(s) 208, etc. Memory 210 can include any suitable volatile memory, non-volatile memory, storage, or any suitable combination thereof. For example, memory 210 can include RAM, ROM, EEPROM, one or more flash drives, one or more hard disks, one or more solid state drives, one or more optical drives, etc. In some embodiments, memory 210 can have encoded thereon a computer program for controlling operation of computing device 110. In such embodiments, processor 202 can execute at least a portion of the computer program to present content (e.g., user interfaces, graphics, tables, reports, etc.), receive genetic data from data source 102, receive information (e.g., content, genetic information, etc.) from server 120, transmit information to server 120, etc.

In some embodiments, server 120 can include a processor 212, a display 214, one or more inputs 216, one or more communications systems 218, and/or memory 220. In some embodiments, processor 212 can be any suitable hardware processor or combination of processors, such as a CPU, a GPU, an MCU, an ASIC, an FPGA, etc. In some embodiments, display 214 can include any suitable display devices, such as a computer monitor, a touchscreen, a television, etc. In some embodiments, inputs 216 can include any suitable input devices and/or sensors that can be used to receive user input, such as a keyboard, a mouse, a touchscreen, a microphone, etc.

In some embodiments, communications systems 218 can include any suitable hardware, firmware, and/or software for communicating information over communication network 108 and/or any other suitable communication networks. For example, communications systems 218 can include one or more transceivers, one or more communication chips and/or chip sets, etc. In a more particular example, communications systems 218 can include hardware, firmware and/or software that can be used to establish a Wi-Fi connection, a Bluetooth connection, a cellular connection, an Ethernet connection, etc.

In some embodiments, memory 220 can include any suitable storage device or devices that can be used to store instructions, values, etc., that can be used, for example, by processor 212 to present content using display 214, to communicate with one or more computing devices 110, etc. Memory 220 can include any suitable volatile memory, non-volatile memory, storage, or any suitable combination thereof. For example, memory 220 can include RAM, ROM, EEPROM, one or more flash drives, one or more hard disks, one or more solid state drives, one or more optical drives, etc. In some embodiments, memory 220 can have encoded thereon a server program for controlling operation of server 120. In such embodiments, processor 212 can execute at least a portion of the server program to transmit information and/or content (e.g., a user interface, graphs, tables, reports, etc.) to one or more computing devices 110, receive genetic data, information, and/or content from one or more computing devices 110, receive instructions from one or more devices (e.g., a personal computer, a laptop computer, a tablet computer, a smartphone, etc.), etc.

FIG. 3 shows an example 300 of a process for determining pathogen-specific adaptive thresholds in accordance with some embodiments of the disclosed subject matter. At 302, process 300 can receive genetic data (e.g., genetic sequencing results) corresponding to one or more negative control samples and/or one or more positive control samples. In some embodiments, the negative controls can includes any suitable genetic data generated from a sample that is expected to not include any pathogens of interest in a clinically significant quantity. Generating negative control results can involve preparing a tube or sample well with all of the reagents used to prepare a clinical sample, but with no sample added. Thus, any reads seen for the tube or sample well corresponding to the negative control cannot be a true signal, and must be attributable to some form of error, such as operator error, contamination (including cross-contamination), ambient labome nucleic acids, some problem with the data-processing pipeline used to generate the final alignments, and/or some other source of error. These errors can be some combination of one-time errors, and errors that recur to varying degrees in each sample run by a laboratory. Note that negative controls can sometimes be referred to as background controls.

In some embodiments, the genetic data received at 302 can include any suitable information, and can be in any suitable format. For example, in some embodiments, the genetic data received at 302 can be formatted as results from a next generation sequencing device. In more particular example, the results can be formatted as a BCL file, which includes information received from the sequencer's sensors (e.g., regarding the luminescence that represent the biochemical signal of the reaction). In such an example, process 300 can include aligning the genetic data received at 302 (e.g., using alignment system 104). In such an example, the data can be converted into another format, such as a FASTQ format, that includes both a called base and a quality score for each position of a read. As another example, the genetic data received at 302 can be received as reads that include a called base and in some cases a quality score for each position of each read. In a more particular example, the results can be formatted a FASTQ file.

As another example, the genetic data received at 302 can be formatted as a raw count of reads associated with various pathogens and/or other organisms, identifying information of a particular pathogen (and/or other organism) and/or group of pathogens/other organisms (e.g., organized at any suitable taxonomic level, which is sometimes referred to herein as a taxon), and/or identifying information of reads associated with the pathogen and/or other organism (e.g., based on a reference sequence, based on a reference sequence with alternates, etc.). Note that the count of reads can be formatted in multiple ways. For example, the count of reads can be formatted as the total reads (which is sometimes referred to as alignments) that align to each pathogen or other organism, including repeats. As another example, the count of reads can be formatted as the count of reads that align uniquely to that pathogen or other organism, excluding reads that were observed multiple times. In some embodiments, the data received at 302 can be organized such that the data is grouped by taxon, and taxons of different taxonomic rank are represented in the data. For example, the data received at 302 can values associated with particular pathogens (e.g., a taxon at a species or subspecies taxonomic level), and other values associated with a group of pathogens (e.g., a taxon at a genus, family, or order taxonomic level).

As yet another example, the genetic data received at 302 can be formatted as a statistical transform of raw counts. For example, the statistical transform can be based on the proportion of the total counts made up by counts associated with a particular pathogen (e.g., a ratio of reads for pathogen x to total reads, a normalized ratio of reads for pathogen x to total reads). As another example, the statistical transform can be based on uniqueness of the alignment (e.g., the value of the statistical transform can be inversely proportional to the number of other species the alignment maps to), its informational complexity and how closely the read maps to a particular reference genome (e.g., the human genome for samples taken from a human). In such an example, reads that are more unique or that are more complex can be associated with higher values from the transform, while reads that map closely to the particular reference genome can be associated with a lower value.

In some embodiments, results associated with a control sample can be identified as being a positive control sample for one or more organisms, and/or a negative control sample for one or more organisms (note that a sample cannot be a positive control sample and a negative control sample for the same organism). For example, in some embodiments, a file name associated with sequencing results of a sample can identify whether the sample is a positive control sample and/or a negative control sample. As another example, a location of sequencing results of a sample can be used to identify whether the sample is a positive control sample and/or a negative control sample. In a more particular example, a folder in a file system (e.g., of computing device 110) can be designated as being associated with negative control samples, while another folder in the file system can be designated as being associated with positive control samples, and yet another folder in the file system can be designated as being associated with positive clinical samples.

In some embodiments, negative control samples and/or positive control samples can be specific to a particular laboratory, specific to a particular type of laboratory, specific to a particular type of sample being analyzed (e.g., blood sample, sputum sample, fecal sample, etc.), etc. For example, process 300 can be executed for a particular laboratory and a particular type of sample, with each negative control sample being run at that laboratory using that type of sample. As another example, process 300 can be executed for a particular type of laboratory and/or laboratories in a particular region that tend to have similar background levels.

At 304, process 300 can generate a model based on one or more results from the control sample or control samples received at 302. In some embodiments, the model can be used to determine a threshold at which each pathogen in a clinical sample is to be considered clinically significant. In some embodiments, process 300 can generate any suitable type of model. For example, process 300 can generate one or more statistical model for various organisms (e.g., pathogens) based on one or more control samples. In such an example, the statistical model can be used to determine an explicit threshold for a particular pathogen (or other organism) at which a clinical sample can be considered clinically significant. In such an example, if a value in results from a clinical sample meets and/or exceeds the threshold for a particular pathogen, that pathogen can be considered positive (i.e., present) in the sample.

As another example, process 300 can generate a machine learning model for various organisms (e.g., pathogens) based on one or more control samples. In such an example, an output of the machine learning model can be indicative of whether a particular pathogen is present in the sample. In such an example, the machine learning model may not generate an explicit threshold in terms of a semantically meaningful value (e.g., raw read count, a statistical transform of raw read counts). However, a threshold may be applied to the output of the machine learning model (e.g., for each pathogen). In a more particular example, the output for each pathogen can be a value in a range [0, 1] (e.g., where higher numbers indicate a higher likelihood of the value indicating the presence of the corresponding pathogen). A threshold can be selected for the output (e.g., at 0.5, 0.75, 0.9, etc.), where an output that is at or above the threshold indicates a positive result for that pathogen, and a value under the threshold indicates a negative result for that pathogen.

Note that in some embodiments, the statistical model at 304 can be generated based on control sample results and/or clinical sample results. For example, a kernel density estimation-based model can be based on clinical sample results.

At 306, process 300 can receive genetic data associated with a clinical sample (e.g., from data source 102, from alignment system 104). In some embodiments, the genetic data can be formatted in any suitable format. For example, the genetic data received at 306 can be formatted in a format described above in connection with 302.

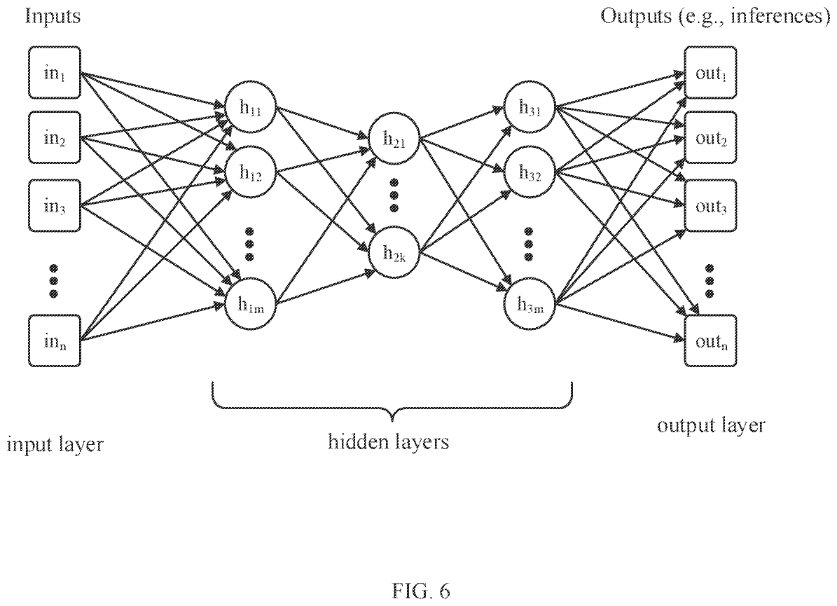

At 308, process 300 can use the model generated at 304 to determine, for each pathogen represented in the clinical sample results (and/or each pathogen of interest), whether the result is likely clinically significant. For example, if the model is used to generate an explicit threshold for various pathogens, process 300 can determine whether the clinical results for a particular pathogen meet or exceed the explicit threshold. As another example, if the model is a machine learning model, the clinical results can be provided as input to the machine learning model (e.g., a neural network) and outputs of the machine learning model can be used to determine a likelihood that each pathogen is clinically significant. In a more particular example, a value associated with a pathogen or group of pathogens can be provided as input to an input node associated with the pathogen or group of pathogens. An output from a corresponding output node can be a prediction of whether the value associated with the pathogen or group of pathogens represents a signal (e.g., the pathogen or one or more pathogens in the group of pathogens is present in the sample) or noise (e.g., the pathogen or one or more pathogens in the group of pathogens is not present in the sample). As described below in connection with FIGS. 6, 9, and 11 to 13C, and TABLES 2 and 3, the output can be formatted as a value in a range of zero to one, with values closer to zero indicating a greater likelihood that the pathogen is not present in the sample, and values closer to one indicating a greater likelihood that the pathogen is present in the sample. As yet another example, if the model is a statistical model based on the clinical sample, process 300 can determine whether a particular pathogen is likely to be clinically significant based on the model (e.g., based on a kernel density estimate, etc.).

At 310, process 300 can generate a report based on the clinical sample results, the one or more determinations made based on the model, and/or the one or more control sample results. In some embodiments, the report can include any suitable content, information, and/or data. For example, the report can include a list of pathogens (if any) that are likely to be clinically significant. As another example, the report can include information indicating confidence in the classification of any positive results. As yet another example, the report can include graphics (e.g., one or more heatmaps, one or more boxplots, etc.) indicative of the results generated for the clinical sample and/or one or more control samples. As still another example, the report can include a list of pathogens that are unlikely to be clinically significant and/or a list of pathogens for which clinical significance is unclear.

At 312, process 300 can cause at least a portion of the report to be presented to a user. For example, in some embodiments, process 300 can cause a computing device (e.g., computing device 110) to present at least a portion of the report to a user. In some embodiments, process 300 can cause the report or a portion thereof to be presented in response to a request. As another example, process 300 can cause the report to be sent to an inbox or other storage location from which the report can be retrieved (e.g., for analysis by a user).

FIG. 4 shows an example 400 of a process for generating a statistical model using negative control samples for determining pathogen-specific adaptive thresholds in accordance with some embodiments of the disclosed subject matter. At 402, process 400 can receive results for one or more negative control samples. In some embodiments, the results can be formatted in any suitable format, such as formats described above in connection with 302 of FIG. 3. In some embodiments, the results for the negative control samples can be results that were generated in a laboratory that is to use the model generated by process 400, or a laboratory that is expected to (or has been shown to) have a similar labome.

At 404, process 400 can generate a distribution for each pathogen and/or other organism from which the sample or samples are believed to be negative. For example, the distribution can be a distribution of raw read counts associated with each negative sample. As another example, the distribution can be distribution of values generated from a statistical transform of raw read counts. Additionally or alternatively, in some embodiments, process 400 can generate a distribution for a group of pathogens and/or other organisms. For example, pathogens and/or other organisms can be grouped based on one or more taxonomic classifications associated with each pathogen and/or other organism (e.g., by species, genus, family, order, class, phylum, and/or kingdom). In such an example, at a relatively low number of negative control samples, pathogens and/or other organisms can be grouped at a high taxonomic level (e.g., kingdom, phylum, etc.), and as the number of negative control samples grows the pathogens and/or other organisms can be grouped at a lower taxonomic level. In such examples, process 400 can generate a distribution for each group. In a more particular example, process 400 can use the distribution for a group to which a pathogen or other organisms belongs in lieu of an individual distribution if process 400 determines that the number of reads associated with that pathogen or organism is insufficient to generate a statistically meaningful distribution.

At 406, process 400 can set a threshold for each pathogen and/or other organism based on the distribution generated at 404. In some embodiments, the threshold can be set at any suitable value based on the distribution. For example, the threshold can be set as the median value of the distribution. As another example, the threshold can be set as the maximum value observed in the distribution. As yet another example, the threshold can be set at two standard deviations over the mean (e.g., for a normal distribution). As still another example, the threshold can be set as at the 95th quartile value from the distribution. As a further example, the threshold can be set as the mean value of the distribution.

In some embodiments, process 400 can assign a threshold for pathogens and/or other organisms of interest for which a threshold cannot be calculated. For example, a threshold may not be able to be calculated because the negative control samples include no results for a particular pathogen and/or other organism, or because the negative control is not classified as being negative for that particular pathogen. In a more particular example, the threshold for such pathogens and/or other organisms can be set to 0, and the threshold can be associated with an indication that the threshold was assigned and not calculated. In another more particular example, the threshold for such pathogens and/or other organisms can be set to a lowest threshold value for a pathogen and/or other organism for which a threshold was calculated. In such an example, the threshold can be associated with an indication that the threshold was assigned and not calculated.

In some embodiments, the thresholds set at 406 can be used by process 300 to determine whether a result is likely to be clinically significant at 308. In some embodiments, an odds ratio can be generated based on a ratio of the result and the threshold. For example, the odds ratio can be the value for a particular pathogen over the threshold value for that pathogen (if such a threshold exists). For example, if the threshold is 1×10\hat{ }-5, and the result is 2×10\hat{ }-5, than the odds ratio can be reported as 2.0. In some embodiments, if a particular pathogen is not associated with an explicit threshold (e.g., because it was not present at all in the negative control samples), the odds ratio can be calculated based on a smallest threshold across all pathogens and/or other organisms for which an explicit threshold was determined. In such embodiments, an indication that the odds ratio is based on an inferred threshold rather than an explicit threshold can be presented to a user.

At 408, process 400 can receive results for one or more additional negative control samples. As described above in connection with 402, the results can be received in any suitable format. In some embodiments, a laboratory can periodically (e.g., at regular and/or irregular intervals, such as daily, weekly, in connection with each clinical sample, in connection with every nth clinical sample, after a triggering event such as a suspected contamination event or a deep cleaning of the laboratory, etc.) run a negative control sample, and generate results for an additional negative control sample.

At 410, process 400 can determine whether the additional negative control results are normal (e.g., within an expected normal distribution), or abnormal (e.g., containing one or more outliers that depart from the previous distribution). In some embodiments, process 400 can use any suitable technique or combination of techniques to determine whether the additional negative control results are normal. For example, process 400 can compare the new results for each pathogen to the previous distribution for that pathogen, and determine whether the new results for the pathogen is within a normal curve fitted to the previous distribution. As another example, process 400 can compare the new results for each pathogen to the previous distribution for that pathogen, and determine whether the new results for the pathogen are lower than a maximum value included in the previous distribution.

At 412, if process 400 determines that the new results are normal (“YES” at 412), process 400 can return to 404 and generate a new and/or updated distribution for setting thresholds. Otherwise, if process 400 determines that the new results are abnormal (“NO” at 412), process 400 can move to 414.

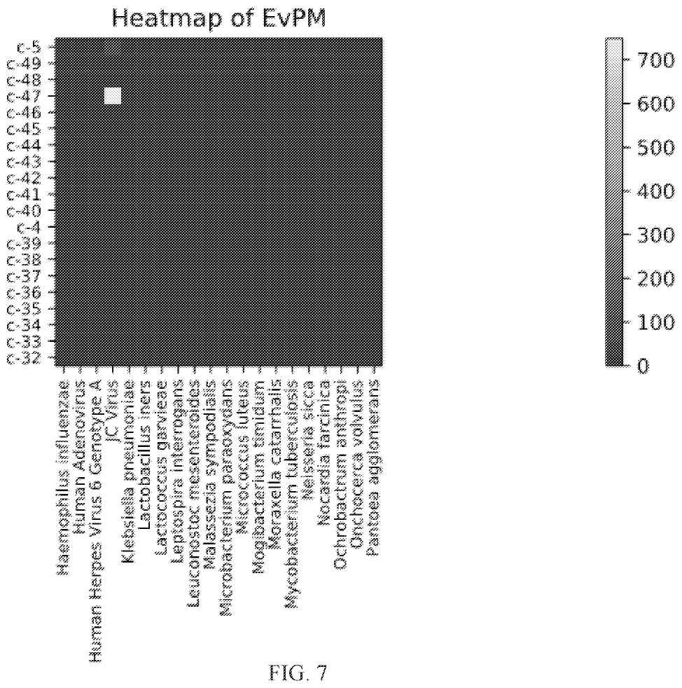

At 414, process 400 can cause the abnormal results to be presented to a user as representing a divergence from expected negative control results. For example, the abnormal results can be presented in connection with the existing results to illustrate for the user how different the new results are from the existing results. In such an example, the abnormal results and the previous results can be plotted as a histogram (e.g., using different styling to distinguish between existing results and new results), as a heat map (e.g., using rows to separate samples as illustrated in FIGS. 6 and 7 described below), and/or using any other suitable visualization.

In some embodiments, process 400 can prompt a user to provide input indicating how to respond to the abnormal results. For example, process 400 can cause a user interface to be presented with user interface elements allowing a user to choose one or more options for addressing the potentially abnormal new results.

At 416, process 400 can receive input indicating whether the new results are user-verified as normal (i.e., to be used in generating a distribution) and/or whether to disregard or otherwise inhibit the use of any results (e.g., new results and/or existing results) in generating a distribution. For example, process 400 can receive input indicating that the new results are to be disregarded for one or more (or all) pathogens and/or other organisms. As another example, process 400 can receive input indicating that the new results are to be used to generate a new distribution, and that the existing results are to be disregarded for one or more (or all) pathogens and/or other organisms. As yet another example, process 400 can receive input indicating that the new results are to be used to as normal results to generate a new and/or updated distribution.

After receiving input indicating how the potentially abnormal new results are to be used (or not used) at 416, process 400 can return to 404 to generate a new and/or updated model based on the input received at 416.

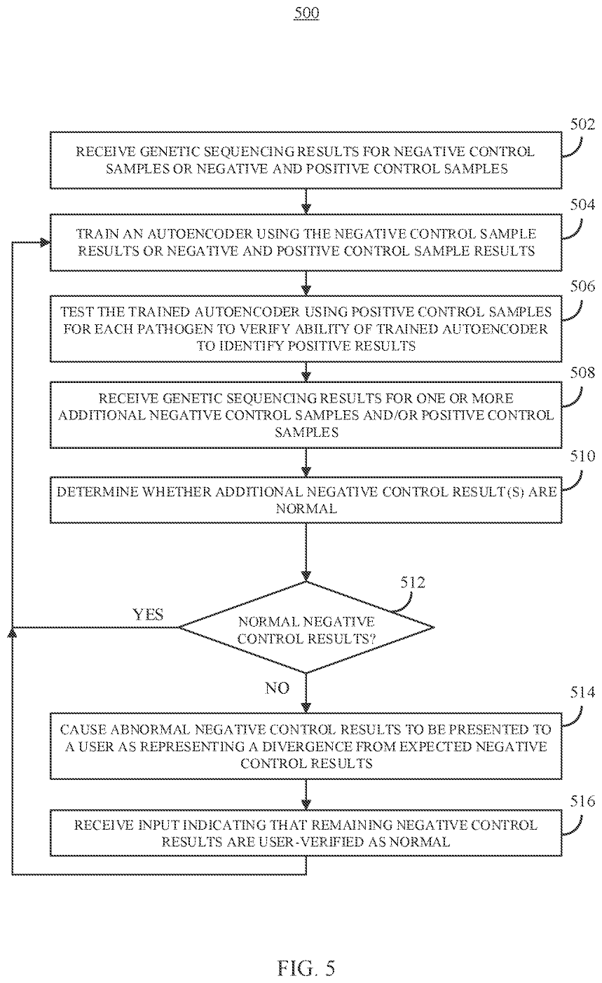

FIG. 5 shows an example of a process for generating a machine learning model using negative control samples or negative control samples and positive control samples for determining pathogen-specific adaptive thresholds in accordance with some embodiments of the disclosed subject matter. At 502, process 500 can receive results for one or more negative control samples or for one or more negative control samples and one or more positive control samples. In some embodiments, the results can be formatted in any suitable format, such as formats described above in connection with 302 of FIG. 3. In some embodiments, the results for the negative control samples and/or the positive control samples can be results that were generated in a laboratory that is to use the model generated by process 500, or a laboratory that is expected to (or has been shown to) have a similar labome. In some embodiments, the positive control samples can be generated based on probit runs spiked with multi-analyte panels, and the results can either include any ancillary results (e.g., pathogens and/or other organisms that were not explicitly spiked in to the sample) or exclude any such ancillary results (e.g., by setting a value associated with any pathogens and/or other organisms that were not spiked in to 0).

At 504, process 500 can train a machine learning model using the negative control sample results or a combination of the negative control sample results and the positive control samples results. In some embodiments, process 500 can train any suitable machine learning model using any suitable technique or combination of techniques. For example, process 500 can train a neural network using the negative control sample results or a combination of the negative control sample results and the positive control samples results to classify future results as being consistent with a clinically significant level of a pathogen, or consistent with a background level of the pathogen. In a more particular example, process 500 can train an autoencoder using the negative control results and the positive control results to represent normal data to be modeled by the autoencoder. As another more particular example, process 500 can train an autoencoder using only negative control results such that autoencoder is trained to represent normal data as negative. In such an example, a scaled version of the output of the model can be compared to the input for each pathogen, and a difference between the input and the output can be indicative of whether the input represents a clinically significant level of a pathogen. In such an example, the autoencoder can be used to detect positive results as anomalies from the expected results because the autoencoder is trained to model negative results and can be expected to perform poorly when attempting to model positive results. In such examples, an autoencoder can be trained using unsupervised learning which does not require explicit labeling of results as representing positive or negative results.

In some embodiments, an autoencoder can have any suitable architecture. For example, the autoencoder can have an input layer and an output layer with a number of nodes equal to the number of pathogens and/or other organisms for which results are to be generated. As another example, the autoencoder can have any suitable number of hidden layers (e.g., encoding and decoding layers), each having any suitable number of nodes, and can have a coding layer with any suitable number of nodes.

In a more particular example, the autoencoder can have one encoding layer and one decoding layer. In such an example, the encoding layer and decoding layer can have the same number of nodes, and can have a fraction of the nodes included in the input and output layers (e.g., in a range including 2% and 15% of the number of nodes in the input and output layers, in a range including 4% and 13%, in a range including 6% and 11%, in a range including 7% and 10% of the number of nodes in the input and output layers, in a range including 8% and 9% of the number of nodes in the input and output layers, etc.). Additionally, in such an example, the coding layer can include any suitable number of nodes, which can be a small fraction of the number of input nodes and output nodes (e.g., in a range including 0.1% and 1.5% of the number of nodes in the input and output layers).

In another more particular example, the autoencoder can include a second encoding layer and a second decoding layer. In such an example, the second encoding and decoding layer can be disposed between the first encoding or decoding layer and the coding layer, and can have a larger fraction of the nodes including in the input and output layers (e.g., in a range including 20% and 40% of the number of nodes in the input and output layers, in a range including 25% and 35% of the number of nodes in the input and output layers, in a range including 30% and 34% of the number of nodes in the input and output layers, etc.).

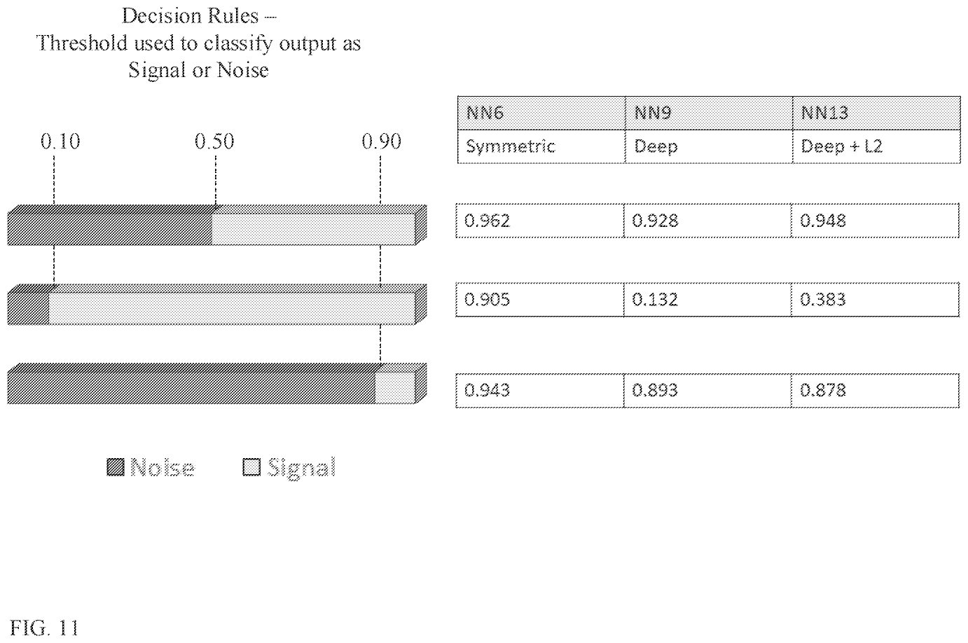

In some embodiments, process 500 can train an autoencoder using any suitable optimizer, loss function, and/or loss metric. For example, process 500 can train an autoencoder using the RMSprop optimizer. As another example, process 500 can train an autoencoder using an Adam optimizer (e.g., based on an optimizer described in Kingma et al., “Adam: A Method for Stochastic Optimization,” available at arxiv(dot)org, 2014). As yet another example, process 500 can train an autoencoder using a mean squared error loss function. As still another example, process 500 can train an autoencoder using a binary-cross-entropy loss function. As a further another example, process 500 can train an autoencoder using accuracy (i.e., how closely the autoencoder represented the input data) as a loss variable for training. In such an example, process 500 can train the autoencoder based on a binary accuracy, in which the output is classified as signal or noise (e.g., based on a threshold, as described below in connection with FIG. 11), and process 500 can determine whether the classification is correct to determine whether the classification is accurate.

In some embodiments, the nodes of the autoencoder can have any suitable activation function. For example, the nodes of each hidden layer can have a rectified linear unit (ReLU) activation function. As another example, the nodes of the output layer can have a sigmoid activation function. In some embodiments, the layers of the autoencoder can be fully connected to each preceding and subsequent layer. Alternatively, one or more layers can be sparsely connected to a preceding and/or subsequent layer.

In some embodiments, one or more techniques can be used to reduce the likelihood that of overfitting by the autoencoder to the training data, such as dropout, regularizing layer weights (e.g., using a linear weight penalty, using a quadratic weight penalty), using early stopping, etc. For example, process 500 can use one or more use dropout techniques during training, in which one or more nodes (e.g., of a particular layer, of each hidden layer, of all hidden layers, etc.) are removed from the network (e.g., during a particular training epoch). In such an example, the probability of each node being removed can be specified (e.g., for a layer, for all hidden layers, for all encoding layers and all decoding layers, etc.), and during a particular epoch, process 500 can randomly determine which nodes are to be removed, and can train the network using the remaining nodes.

As another example, process 500 can use a penalization term in the loss function, such as a linear weight penalty (sometimes referred to as L1 regularization), or a quadratic weight penalty (sometimes referred to as L2 regularization). In such an example, the loss function can be augmented using the weight penalization term, which can discourage the network from using large weights.

As yet another example, process 500 can use an early stopping condition to attempt to inhibit overfitting. For example, process 500 can stop training when 100 training epochs have been completed. As another example, process 500 can stop training when an improvement in accuracy (e.g., as reflected by the loss value) has not improved by at least a threshold amount in a particular number of epochs (e.g., has not substantially improved in the previous five epochs). As yet another example, process 500 can stop training when the loss has not changed over a given number of epochs.

In some embodiments, for example, as described below in connection with FIG. 11, process 500 can train the network using different predictive thresholds that determine which class an output is associated with. For example, in a network configured to perform binary classification, process 500 can train the network using a predictive threshold of 0.5 (e.g., if the output of an output layer is at least 0.5, the output can be classified as positive), and one or more other values (e.g., 0.1, and 0.9; 0.25 and 0.75, etc.).

In some embodiments, process 500 can adjust the structure of the neural network based on pilots at various network sizes. For example, process 500 can train networks (e.g., autoencoders) with various structures (e.g., chosen automatically, based on user input, etc.), and evaluate performance of each network after a particular number of epochs (e.g., 10, 20, 50, etc.), and select the best performing network(s) for further training.

At 506, process 500 can test the trained machine learning model (e.g., a trained autoencoder) using positive control samples that were not included in the training data to verify the ability of the trained machine learning model to correctly classify positive results. In some embodiments, the positive control samples can be actual samples that are run by a laboratory for which the autoencoder has been trained. Additionally or alternatively, the positive control samples can be simulated samples in which genetic data from a reference sequence is randomly inserted into the results (e.g., prior to alignment) at particular values to be used to verify how the trained autoencoder behaves in response to known positive results representing various read counts.

In some embodiments, the machine learning model trained at 504 and tested at 506 can be used by process 300 to determine whether a result is likely to be clinically significant at 308.

At 508, process 500 can receive results for one or more additional negative control samples and/or positive control samples. As described above in connection with 502, the results can be received in any suitable format. In some embodiments, a laboratory can periodically (e.g., at regular and/or irregular intervals, such as daily, weekly, in connection with each clinical sample, in connection with every nth clinical sample, after a triggering event such as a suspected contamination event or a deep cleaning of the laboratory, etc.) run a negative control sample, and generate results for an additional negative control sample. Additionally or alternatively, a laboratory can periodically (e.g., at regular and/or irregular intervals, such as daily, weekly, in connection with each clinical sample, in connection with every nth clinical sample, after a triggering event such as a suspected contamination event or a deep cleaning of the laboratory, etc.) run a positive control sample, and generate results for an additional positive control sample.

At 510, process 500 can determine whether the additional negative control results are normal (e.g., within an expected normal distribution), or abnormal (e.g., containing one or more outliers that depart from the previous distribution). In some embodiments, process 500 can use any suitable technique or combination of techniques to determine whether the additional negative control results are normal. For example, process 500 can compare the new results for each pathogen to the previous distribution for that pathogen, and determine whether the new results for the pathogen is within a normal curve fitted to the previous distribution. As another example, process 500 can compare the new results for each pathogen to the previous distribution for that pathogen, and determine whether the new results for the pathogen are lower than a maximum value included in the previous distribution.

At 512, if process 500 determines that the new results are normal (“YES” at 512), process 500 can return to 504 and train a new and/or updated machine learning model for classifying results. Otherwise, if process 500 determines that the new results are abnormal (“NO” at 512), process 500 can move to 514.

At 514, process 500 can cause the abnormal results to be presented to a user as representing a divergence from expected negative control results. For example, the abnormal results can be presented in connection with the existing results to illustrate for the user how different the new results are from the existing results. In such an example, the abnormal results and the previous results can be plotted as a histogram (e.g., using different styling to distinguish between existing results and new results), as a heat map (e.g., using rows to separate samples as illustrated in FIGS. 6 and 7 described below), and/or using any other suitable visualization.

In some embodiments, process 500 can prompt a user to provide input indicating how to respond to the abnormal results. For example, process 500 can cause a user interface to be presented with user interface elements allowing a user to choose one or more options for addressing the potentially abnormal new results.

At 516, process 500 can receive input indicating whether the new results are user-verified as normal (i.e., to be used in generating a distribution) and/or whether to disregard or otherwise inhibit the use of any results (e.g., new results and/or existing results) in generating a distribution. For example, process 400 can receive input indicating that the new results are to be disregarded for one or more (or all) pathogens and/or other organisms. As another example, process 500 can receive input indicating that the new results are to be used to generate a new distribution, and that the existing results are to be disregarded for one or more (or all) pathogens and/or other organisms. As yet another example, process 500 can receive input indicating that the new results are to be used to as normal results to generate a new and/or updated distribution.

After receiving input indicating how the potentially abnormal new results are to be used (or not used) at 516, process 500 can return to 504 to train a new and/or updated machine learning model based on the input received at 516.

FIG. 6 shows an example of a topology of an autoencoder that can be generated to predict pathogen-specific adaptive thresholds using mechanisms described herein in accordance with some embodiments of the disclosed subject matter. In generally, an autoencoder can include an input layer, one or more hidden layers, and an output layer (generally having the same number of nodes as the input layer). Each layer of an autoencoder can be fully connected. For example, as shown in FIG. 6, each node in the input layer is connected to each node in the first hidden layer, and each node in the first hidden layer is connected to each node in the next hidden layer, etc. In some embodiments, an autoencoder trained using mechanisms described herein can include an input node associated with each organism (e.g., pathogen) or group of organisms grouped at any suitable taxonomic level (or levels). For example, each input node can correspond to a particular species or sub-species (or any other suitable taxonomic grouping at or below genus), or a variant within a species or subspecies (e.g., a strain). As another example, each input node can correspond to a particular genus. As yet another example, input nodes can correspond to different taxonomical groupings. In a more particular example, some input nodes can correspond to a species, other input nodes can correspond to a sub-species, and yet other nodes can correspond to a genus.

In some embodiments, the autoencoder can be trained with any suitable number of input nodes corresponding to any suitable organisms of interest. For example, as described below in connection with TABLE 2, the input layer can include thousands of input nodes. In a more particular example, the number of input nodes n represented in FIG. 6 can be over 1,000 input nodes, over 2,000 input nodes, over 3,000 input nodes, over 4,000 input nodes, over 5,000 input nodes, etc., with each node representing a particular pathogen or group of pathogens.

As another example, as described below in connection with TABLE 3, the input layer can include fewer than 1,000 input nodes (e.g., in a range including 100 and 900 nodes, in a range including 200 and 800 nodes, in a range including 300 and 700 nodes, in a range including 400 and 600 nodes, in a range including 450 and 550 nodes). In a more particular example, the input layer can include 512 nodes.

In some embodiments, the autoencoder can be configured to include an output node corresponding to each input node. For example, each output node can correspond to a particular organism or group of organisms, and an output can correspond to a prediction of whether that organism is present in a sample.

The relatively simple topology shown in FIG. 5 includes an input layer, three symmetric hidden layers (having m, k, and m nodes, respectively), and an output layer. For example, the input layer can include n input nodes that are configured to receive a floating point input (e.g., representing a raw read count associated with a particular pathogen or group of pathogens, or a statistical transform of such a raw read count), a first hidden layer can include m nodes that are each connected to an output of every input node, a second hidden layer (which is sometimes referred to herein as a coding layer) can include k nodes that are connected to an output of every node in the first hidden layer, were k is less than m and less than n. A third hidden layer can include m nodes that are each connected to an output of every node in the coding layer (note that hidden layers that precede the coding layer are sometimes referred to as encoding layers, and hidden layers that follow the coding layer are sometimes referred to as decoding layers). An output layer can include n output nodes that are each connected to every node in the third hidden layer, and each can be configured to output a value that predicts whether the value provided at the corresponding input node exceeds a threshold. As described below in connection with TABLE 3, an encoder can be configured asymmetrically (e.g., with more hidden layers on one side of the coding layer than the other).

FIG. 7 shows an example of a heatmap presenting sequencing and alignment results for various pathogens from a variety of clinical samples in in accordance with some embodiments of the disclosed subject matter. As shown in FIG. 7, a heatmap can be generated that is coded based on a value representing a raw read count for various samples (e.g., a raw read count or a statistical transformation of a raw read count). In the example shown in FIG. 7, the values are plotted based on a statistical transform of raw read count for a particular pathogen. In particular the values in FIG. 7 are based on a statistical transform of the number of reads that is based on a normalized ratio of reads for each pathogen to total reads, which is referred to in the heatmap as evidence per million. Note that the results shown in FIG. 7 are clinical results, with a single result (JC virus for sample c-47) being strongly positive, while the rest have low values (e.g., in a range from 0-200).

In some embodiments, results for a particular clinical sample can be presented with other clinical results (e.g., anonymized clinical results) and/or control sample results, to provide a relatively intuitive graphic that a user can use to interpret results. In some embodiments, results can be sorted and/or grouped based on a result of a model used to determine a likelihood that a particular result is clinically significant. For example, if the results received at 306 include information about the relative abundance of genetic information associated with several thousand different pathogens, process 300 can be used to determine which results are likely to be most important (e.g., results that are potentially positive), and results that can be de-emphasized (e.g., results that are less likely to be positive). Results that are more likely to be positive can be presented in a prominent position (e.g., near a beginning of a heat map), while results that are less likely to be positive can be relegated to a less prominent position (e.g., closer to the end of the heat map, grouped with other similar pathogens, etc.).

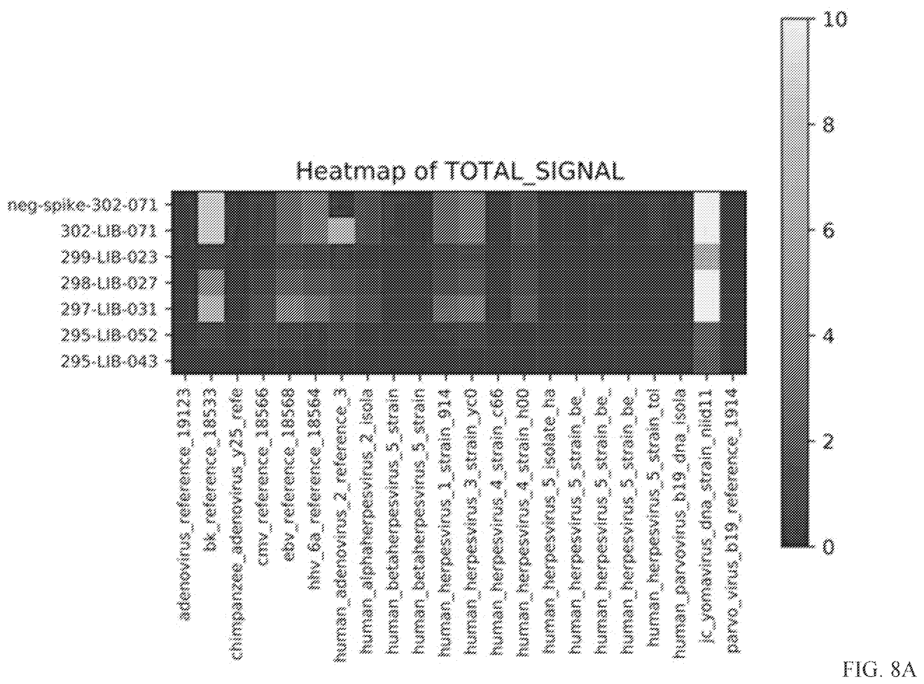

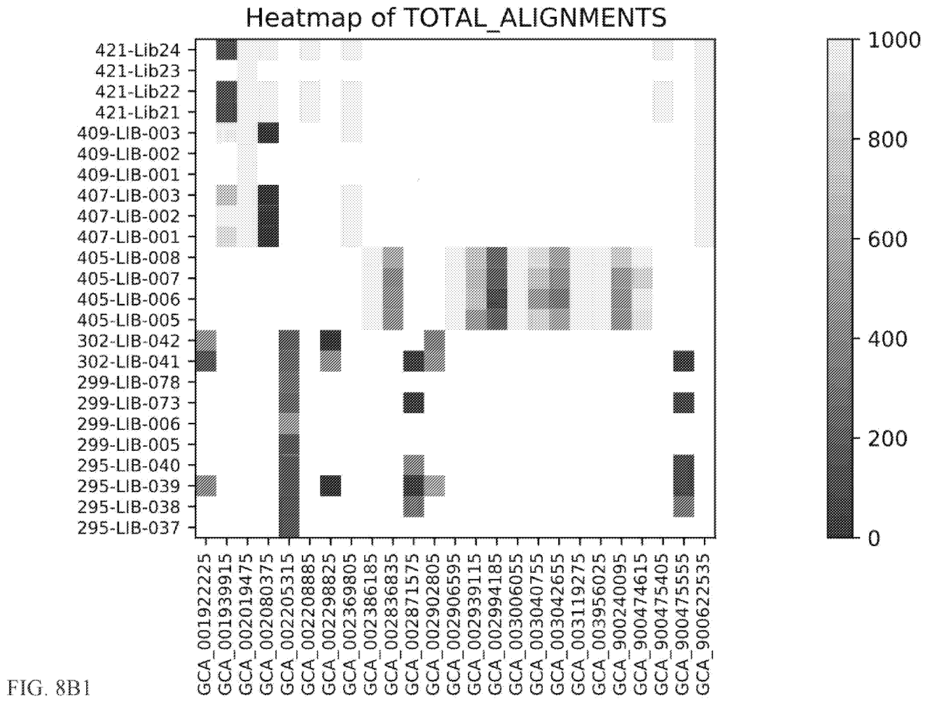

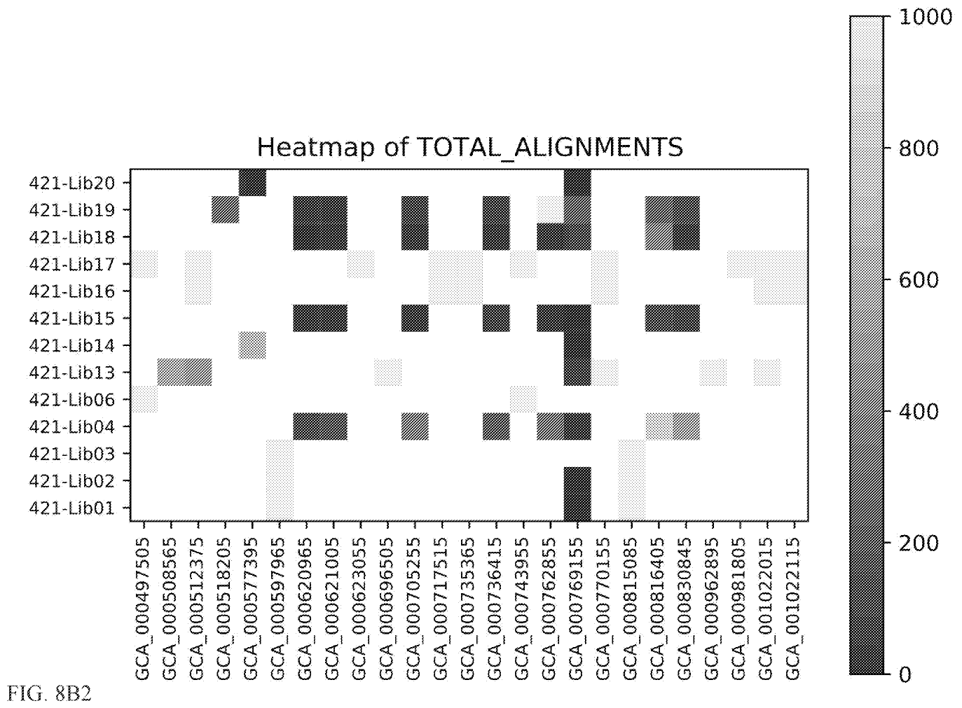

FIG. 8A shows an example of a heatmap presenting sequencing and alignment results for various pathogens from a variety of positive control samples used to test models generated in accordance with some embodiments of the disclosed subject matter, and FIGS. 8B1 and 8B2 show examples of heatmaps presenting sequencing and alignment results for various pathogens from a variety of negative control samples used to test models generated in accordance with some embodiments of the disclosed subject matter. The results illustrated in the heat map were positive control samples generated by spiking in varying amounts (in units/millimeter (ml)) of various pathogens from a group of 57 positive control samples used to generate various models for determining pathogen-specific adaptive thresholds in accordance with some embodiments of the disclosed subject matter. The amount spiked in is included in TABLE 1.

TABLE 1

| Sample | Sample | units/ml spiked in | Notes |
|---|---|---|---|
| 1 | 295-LIB-043 | 75 | |
| 2 | 295-LIB-052 | 150 | |
| 3 | 299-LIB-023 | 300 | |
| 4 | 298-LIB-027 | 1200 | |
| 5 | 297-LIB-031 | 10,000 | |
| 6 | 302-LIB-071 | 10,000 | |
| 7 | NEG-SPIKE-302-071 | 10,000 | Copy of NEG spike 302-071 |
| | | | with ADV signal set to 0.0 |

In the example shown in FIG. 8A, the values are plotted based on a statistical transform of raw read count for a particular pathogen. In particular the values in FIG. 8A are based on uniqueness of the alignment, its complexity, and how closely the read maps to the human genome (referred to as “Signal” in FIG. 8A). As shown in FIG. 8A, as the concentration of spiked in sample increases, the value shown in the het map generally increases. However, certain samples (e.g., 299-LIB-023) that had relatively low concentrations spiked in appear to be due to contamination or another error due to the low level present. Accordingly, even an expert may erroneously believe that such a result is negative. Note that the results in FIG. 8A are for particular reference sequences and/or strains of a pathogen, whereas the results in FIG. 7 are more generalized. Mechanisms described herein can selectively present information in any suitable level of detail based on the level of detail desired by a user and/or based on which results are determined to be most useful.

In the examples shown in FIGS. 8B1 and 8B2, the values are plotted based on raw read count for a particular pathogen (referred to as Total_Alignments in FIGS. 8B1 and 8B2). As shown in FIGS. 8A and 8B, many of the same organisms are present in the negative control samples in similar amounts, although the specific pathogens and concentrations changes to some extent over time. Note that the labels for the x-axis are associated with reference files that are drawn from assemblies of various pathogens.

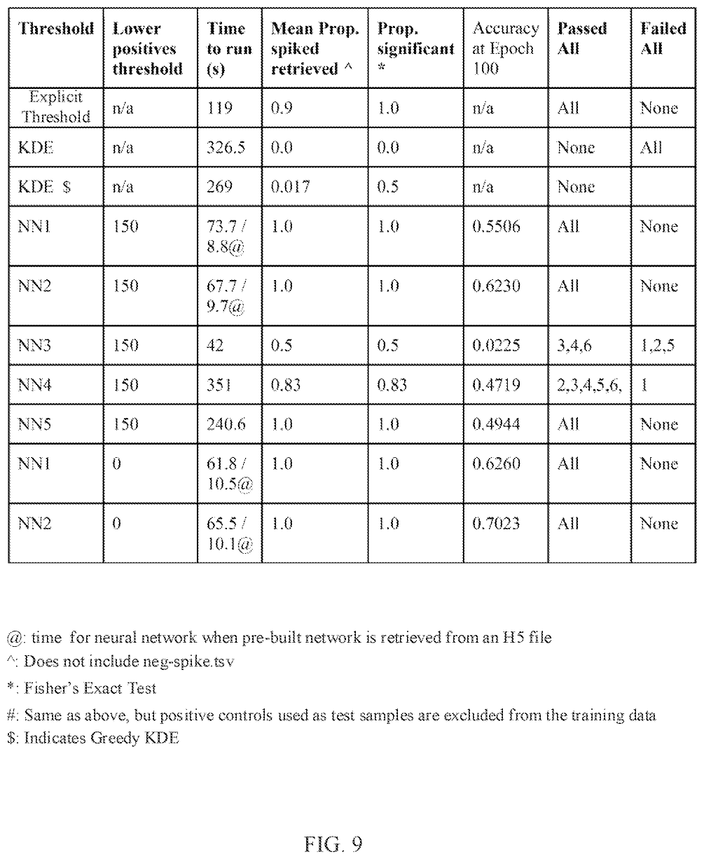

FIG. 9 shows an example of a table presenting classification results for the variety of simulated samples generated by various models in accordance with some embodiments of the disclosed subject matter. The results in FIG. 9 represent results of various models in successfully classifying the positive controls described in TABLE 1 and shown in the heatmap of FIG. 8.

The Explicit Threshold model was implemented in accordance with techniques described above in connection with FIG. 4 in which a distribution of negative control sample results is used to generate a threshold for various pathogens. In the results of FIG. 9, 32 negative control samples were used to generate distributions, and thresholds were set at the median of the distribution. As shown in FIG. 9, the Explicit Threshold model successfully classified each positive sample shown in FIG. 8 as positive. In FIG. 9, the column Passed All is used to note which of the positive control samples in TABLE 1 the model correctly classified all 12 of the spiked-in pathogens as being positives (i.e. 12 of the 12 pathogens spiked in were identified as being likely to be diagnostically relevant). Similarly, the column Failed All is used to note which of the positive control samples in TABLE 1 the model incorrectly classified all 12 of the spiked-in pathogens as being negatives (i.e., none of the 12 pathogens were identified as being likely to be diagnostically relevant). Where cells in these columns contain numbers, the numbers refer to the number of the tittered sample from TABLE 1 for which the model either correctly or incorrectly classified all of the pathogens.

The KDE models were implemented using a kernel density estimation on the distribution of results. Since the value for each pathogen for each sample can be represented by a single number (e.g., raw count, a proportional count such as evidence per million referenced in FIG. 7, or a value based on a more complex statistical transform such as Signal referenced in FIG. 8A), each result is one-dimensional for each pathogen. The values of the negative controls, positives controls and clinical samples for a particular pathogen can all be plotted together on the x-axis while the density of that signal is plotted on the y-axis. That signal is mapped onto a kernel (epanechnikov, tophat, and gaussian are among the options in the software) and the breaks (i.e., local minima) of the distribution are marked. From there, KDE can operate in two modes. Normal mode takes everything less than the lowest break as negative, and everything above the highest break as positive, while everything else is left undecided. Greedy KDE (marked with a $ in FIG. 9) allows a maximum of one central bin between breaks to be classed as undecided, with everything else classed as either negative or positive based on their signal relative to that central bin or the central break if no central bin is present (this would happen with, for example, three breaks and thus four bins). As shown in FIG. 9, the KDE models did not successfully classify the results, and required very long runtimes compared to the other models.

The NN models described below in TABLE 2 were all autoencoders trained on the 32 negative control samples used to generate the Explicit Threshold model and 57 positive control samples, including the positive control samples shown in FIG. 8 used for testing unless otherwise noted. The autoencoders had architectures described below in connection with TABLE 2, and were trained in accordance with techniques described above in connection with FIG. 5.

TABLE 2

| Name | Input Layer | Output Layer | Hidden Layers |
|---|---|---|---|
| NN1 | 5961 Floats | 5961 Booleans | 363 nodes, weights 0-1 |
| | | (Sigmoid 0-1) | 10 nodes, weights 0-1 - coding layer |
| | | | 363 nodes, weights 0-1 |
| NN2 | 5961 Floats | 5961 Booleans | 363 nodes, weights 0-1 |
| | | (Sigmoid 0-1) | Dropout layer (0.5) |
| | | | 2000 nodes, weights 0-1 |
| | | | 10 nodes, weights 0-1 - coding layer |
| | | | 2000 nodes, weights 0-1 |
| | | | Dropout layer (0.5) |
| | | | 363 nodes, weights 0-1 |
| NN3 | 5961 Floats | 5961 Booleans | 10 nodes, weights 0-1 - coding layer |
| | | (Sigmoid 0-1) | |
| NN4 | 5961 Floats | 5961 Booleans | 11922 nodes, weights 0-1 |
| | | (Sigmoid 0-1) | |
| NN5 | 5961 Floats | 5961 Booleans | 11922 nodes, weights 0-1 |
| | | (Sigmoid 0-1) | 10 nodes, weights 0-1 - coding layer |

Note that NN1 was trained twice, once using all 32 negative control samples and all positive controls with a concentration of at least 150 units/ml, and once using all 32 negative control samples and all positive controls. NN2 was also trained twice, once using all 32 negative control samples and all positive controls with a concentration of at least 150 units/ml, and once using all 32 negative control samples and all positive controls.

As shown in FIG. 9, NN1 and NN2 performed comparably to the Explicit Threshold model, but required less time to run. The time before the slash is the time for the neural network to be created for the first time and then run, whereas the time after the slash is the time taken when the network is loaded from a file. Note that files can be loaded and then progressively trained.

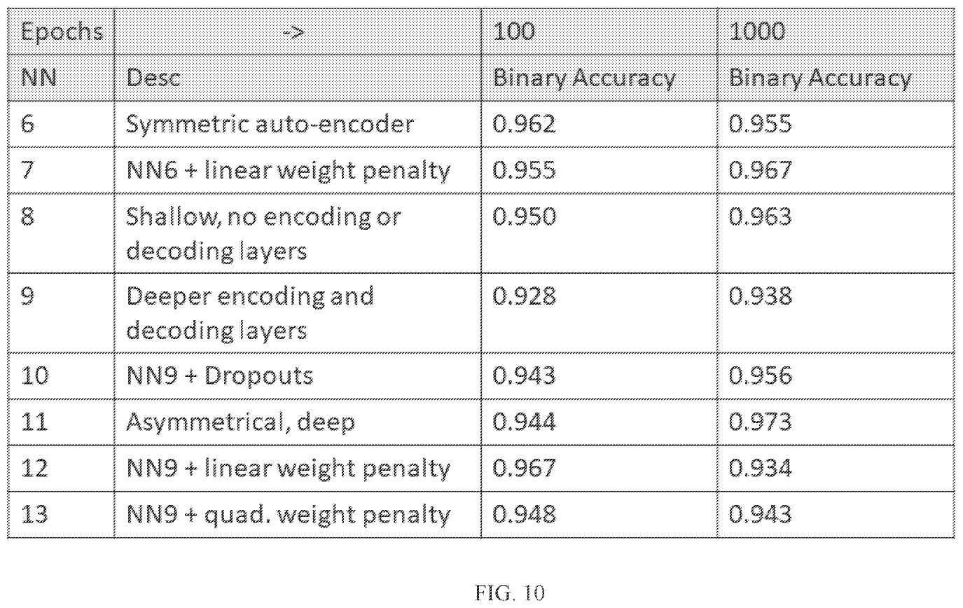

FIG. 10 shows an example of a table presenting classification results for a variety of samples generated by various models trained for different lengths of time in accordance with some embodiments of the disclosed subject matter. The results in FIG. 10 represent results of various models in successfully classifying positive controls shown in the heatmap of FIGS. 8A to 8B2, and negative controls.

The NN models described below in TABLE 3 are autoencoders trained on negative control samples and positive control samples, including positive control samples shown in FIG. 8. The architectures of the autoencoders are described below in connection with TABLE 3, and were trained in accordance with techniques described above in connection with FIG. 5. The NN models described in TABLE 3 were trained using an Adam optimizer (e.g., based on an optimizer described in Kingma et al., “Adam: A Method for Stochastic Optimization,” available at arxiv(dot)org, 2014), and a binary cross entropy loss function. As described below in connection with FIG. 10, the NN models can be trained for a variable number of epochs (e.g., at least 100 epochs, up to 1,000 epochs). As described below in connection with FIG. 11, the NN models can be trained using different decision rules to classify an output of each output node (e.g., classifying an output as “signal” if the output is greater than 0.1, 0.5, or 0.9). Unless otherwise specified, weights associated with hidden layers can have values between 0 and 1. The hidden layers are generally implemented using a rectified linear unit activation function. TABLE 4 shows libraries from which training (sometimes referred to herein as derivation), validation, and testing samples were derived. The libraries included 120 samples of which 37 were known negatives and the rest were probit samples with initial spiked in concentrations of between 1 and 10,000 copies per milliliter of the following pathogens: Human mastadenovirus B, Human parvovirus B19, Human polyomavirus 1 strain BK, Human cytomegalovirus, Epstein-Barr Virus, Human Herpes Virus 6A, Human Herpes Virus 6B, Human alphaherpesvirus 1, Human alphaherpesvirus 2, Human polyomavirus 1 strain JC, Varicella-zoster virus. Twenty percent of the samples were randomly selected for inclusion in a test cohort, and the other 80% of samples were used as a derivation cohort (including 60% of the sample) and a validation cohort (including 20% of the samples) used during derivation after each epoch to evaluate the accuracy of the model. The test cohort was used to evaluate accuracy of the model after training was complete. The results in FIG. 10 are based on predictions of the test cohort.

TABLE 3

| | Input | Output | |
|---|---|---|---|
| Name | Layer | Layer | Hidden Layers |
| NN6 | 512 Floats | 512 | 64 nodes, ReLU activation |
| | | (Sigmoid 0-1) | 32 nodes, ReLU activation |
| | | | 64 nodes, ReLU activation |
| NN7 | 512 Floats | 512 | 64 nodes, ReLU activation |
| | | (Sigmoid 0-1) | 32 nodes, ReLU activation, and Keras Layer 1 |
| | | | (linear) regularizer |
| | | | 64 nodes, ReLU activation |
| NN8 | 512 Floats | 512 | 32 nodes, ReLU activation |
| | | (Sigmoid 0-1) | |
| NN9 | 512 Floats | 512 | Input/2 nodes, ReLU activation |
| | | (Sigmoid 0-1) | 64 nodes, ReLU activation |
| | | | 32 nodes, ReLU activation |
| | | | 64 nodes, ReLU activation |
| | | | Input/2 nodes, ReLU activation |
| NN10 | 512 Floats | 512 | Input/2 nodes, ReLU activation, dropout 0.2 |
| | | (Sigmoid 0-1) | 64 nodes, ReLU activation |
| | | | 32 nodes, ReLU activation |
| | | | 64 nodes, ReLU activation |
| | | | Input/2 nodes, ReLU activation, dropout 0.2 |
| NN11 | 512 Floats | 512 | Input/2 nodes, ReLU activation, dropout 0.2 |
| | | (Sigmoid 0-1) | 64 nodes, ReLU activation |
| | | | 32 nodes, ReLU activation, and Keras Layer 1 |
| | | | (linear) regularizer |
| NN12 | 512 Floats | 512 | Input/2 nodes, ReLU activation, and Keras |
| | | (Sigmoid 0-1) | Layer 1 (linear) regularizer |
| | | | 64 nodes, ReLU activation |
| | | | 32 nodes, ReLU activation |
| | | | 64 nodes, ReLU activation |
| | | | Input/2 nodes, ReLU activation, and Keras |
| | | | Layer 1 (linear) regularizer |
| NN13 | 512 Floats | 512 | Input/2 nodes, ReLU activation, and Keras |
| | | (Sigmoid 0-1) | Layer 2 (quadratic) regularizer |
| | | | 64 nodes, ReLU activation |
| | | | 32 nodes, ReLU activation |
| | | | 64 nodes, ReLU activation |
| | | | Input/2 nodes, ReLU activation, and Keras |
| | | | Layer 1 (quadratic) regularizer |

TABLE 4

| Samples | units/ml spiked in | Notes |
|---|---|---|
| 295; 297, | 0, 1, 20, 40, 150, | Negative background and tittered |
| 298; 299; 302 | 300, 1,200, 10,000 | spiked-in positives (probit samples |
| | | described above) |
| 405; 407; | NA (background) | Deep sequences negative backgrounds |
| 413; 416 | | of asymptomatic human blood plasma |

As shown in FIG. 10, performance of the NN models described above in connection with TABLE 3 was generally comparable after 100 epochs and 1,000 epochs.

FIG. 11 shows an example of a table presenting classification results for a variety of samples generated by various models using different decision rules in accordance with some embodiments of the disclosed subject matter. The results in FIG. 11 represent results of various models in successfully classifying positive controls similar to the controls shown in the heatmap of FIG. 8, but using different decision rules to determine whether an output is counted as a positive result (e.g., a prediction by the model that the input value represents a true positive) or negative result (e.g., a prediction by the model that the input value represents a true negative). The three NN models in FIG. 11 were trained using a threshold of 0.10, 0.50, and 0.90 to classify outputs of the output nodes. For example, if an output node generates a value of at least 0.11 (or 0.10 if the threshold is satisfied by a value that is equal to or greater than the threshold), the NN model prediction for the pathogen or group of pathogens corresponding to the output node (e.g., a pathogen or pathogens for which an input was provided to the corresponding input node) is a predicted positive using a threshold of 0.10. As another example, if an output node generates a value of no greater than 0.09 (or 0.10 if the threshold is only satisfied by a value that is greater than the threshold), the NN model prediction for the pathogen or group of pathogens corresponding to the output node (e.g., a pathogen or pathogens for which an input was provided to the corresponding input node) is a predicted negative using a threshold of 0.10. During training, the accuracy of the prediction can be evaluated by determining whether the prediction matches the sample input. For example, the sample does include the pathogen, and the model predicts that the sample is positive for that pathogen, the output can be considered accurate (e.g., correct). As shown in FIG. 11, the NN models trained using a threshold of 0.50 generally exhibited superior accuracy.

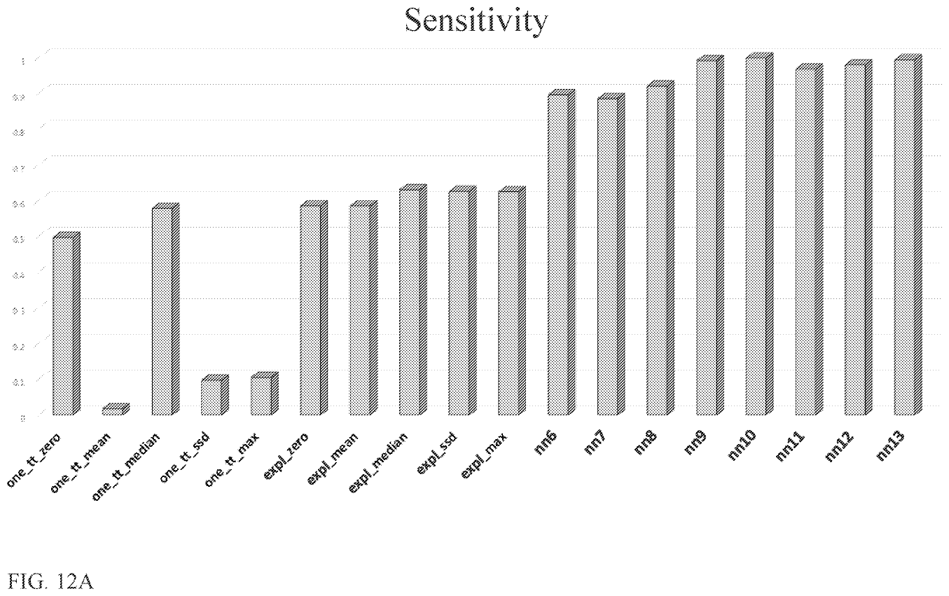

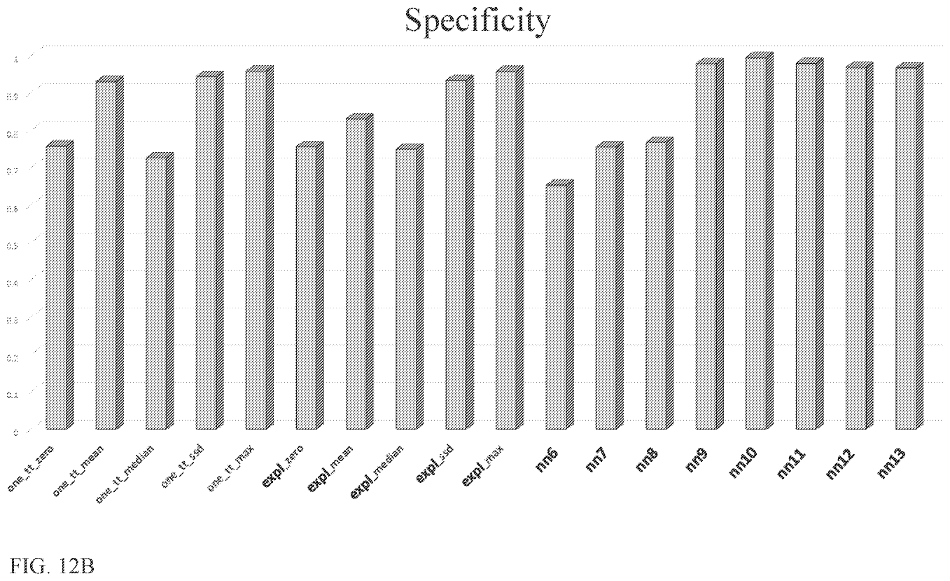

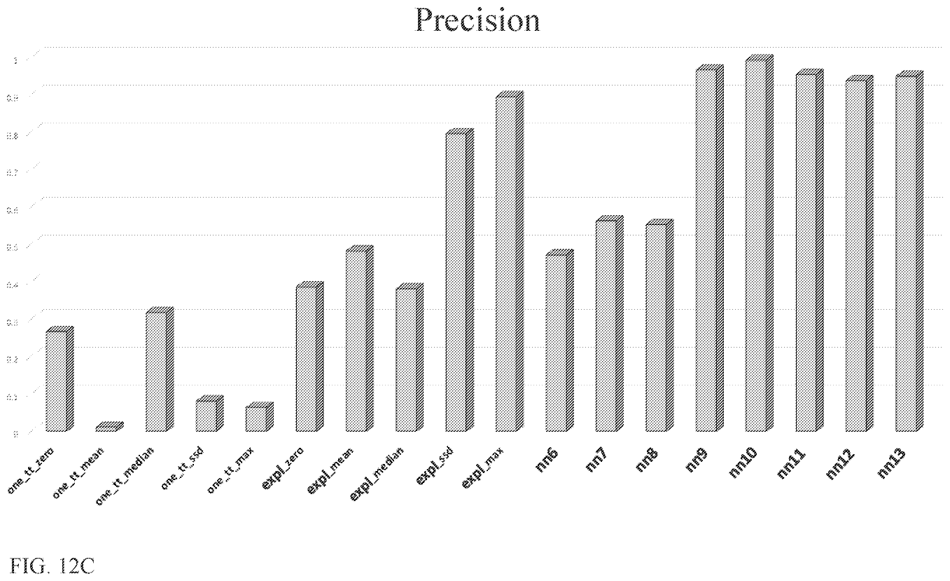

FIGS. 12A to 12C show examples of graphs of sensitivity, specificity, and precision of various techniques used to classify a result as signal or noise in accordance with some embodiments of the disclosed subject matter. The graphs in FIGS. 12A to 12C show comparisons of performance of NN models NN6 to NN13, an explicit threshold model (labeled “expl_ . . . ”) that was implemented in accordance with techniques described above in connection with FIG. 4 in which a distribution of negative control sample results is used to generate a threshold for various pathogens, and a simpler threshold (labeled “one_tt . . . ”) with a threshold calculation performed across pathogens (the “tt” models).

A binary classifier can be evaluated using various metrics. For example, a binary classifier can be evaluated based on how successfully the classifier correctly identifies a positive (e.g., how often the classifier produces a true positive as a fraction of all positive samples), which is sometimes referred as sensitivity. Sensitivity can be calculated by dividing the number of true positives (TP) by the sum of TP and the number of false negatives (FN), which should equal the total number of positives in the test cohort. Sensitivity can be expressed using the relationship: Sensitivity=TP/(TP+FN).

As another example, a binary classifier can be evaluated based on how successfully the classifier correctly identifies a negative (e.g., how often the classifier produces a true negative as a fraction of all negative samples), which is sometimes referred as specificity. Specificity can be calculated by dividing the number of true negatives (TN) by the sum of TN and the number of false positives (FP), which should equal the total number of negatives in the test cohort. Specificity can be expressed using the relationship Specificity=TN/(TN+FP).

As yet another example, a binary classifier can be evaluated based on how successfully the classifier avoids incorrectly identifying a positive as a negative (e.g., how often the classifier produces a true positive as a fraction of all positive classifications). Precision can be calculated by dividing TP by the sum of TP and FP, which can represent the rate at which the classifier produces true positives. Precision can be expressed using the relationship: Precision=TP/(TP+FP).

A confusion matrix used to evaluate the classifiers in FIGS. 12A to 12C is shown in TABLE 4. As shown in TABLE 4, an output was considered a true positive if the sample was spiked with a pathogen that was predicted to be present by the classifier, and was considered a false negative positive if the sample was spiked with a pathogen that was not predicted to be present by the classifier. Similarly, an output was considered a true negative if the sample was not spiked with a pathogen that was not predicted to be present by the classifier, and was considered a false positive if the sample was not spiked with a pathogen that was predicted to be present by the classifier.

As shown in FIGS. 12A to 12C, the NN models general had higher sensitivity than the other classifiers, and NN models NN9 to NN13 had specificity as high or higher than the best performing non NN classifier, while NN models NN6 to NN8 had substantially lower specificity. The precision exhibited by NN models NN9 to NN13 was also as high or higher than the best performing non NN classifier, while NN models NN6 to NN8 had substantially lower precision.

TABLE 4

| | | Not |
|---|---|---|
| | Spiked | Spiked |
| Signal | True | False |
| | Positive | Positive |
| Noise | False | True |
| | Negative | Negative |

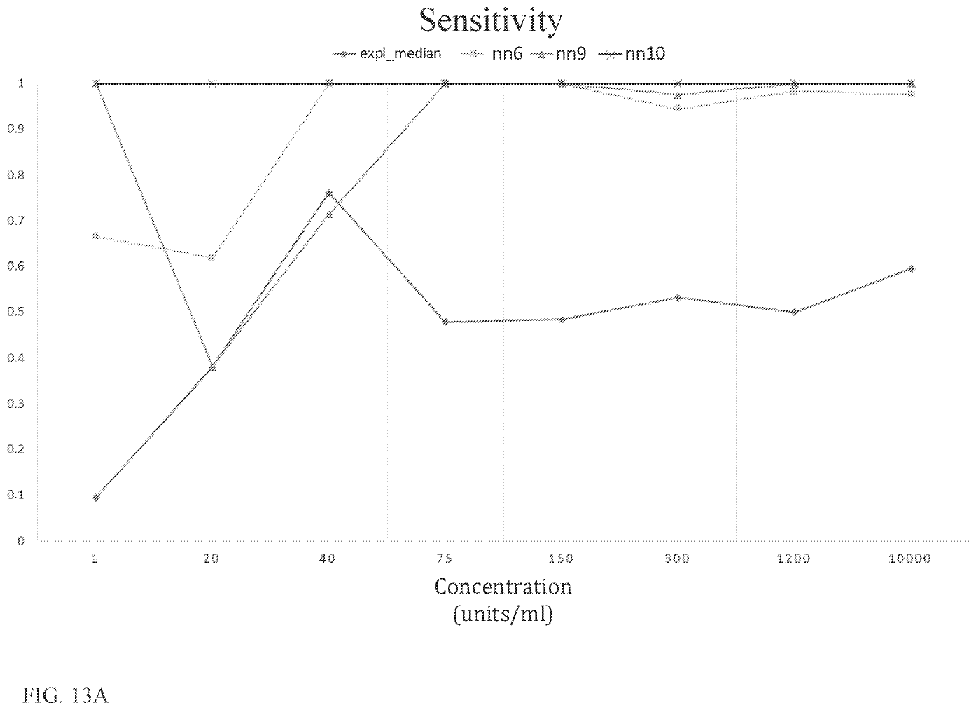

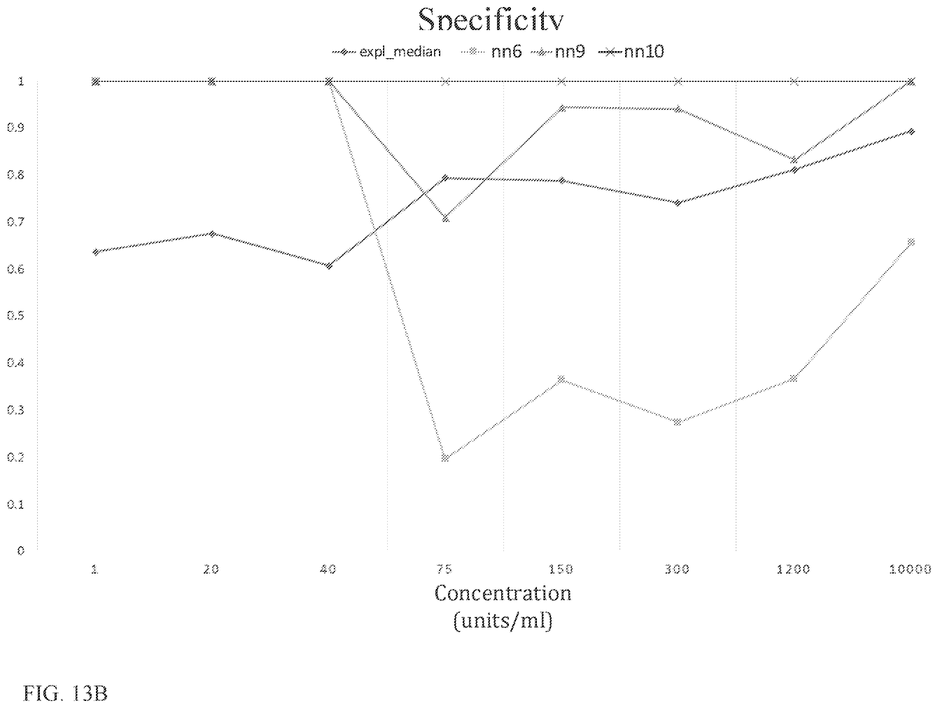

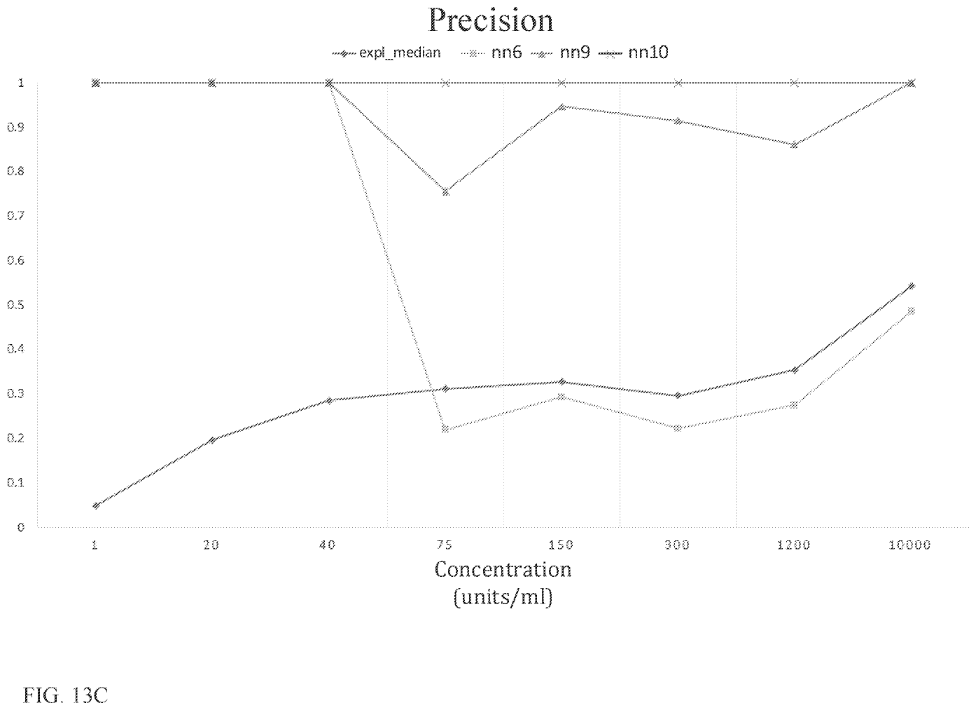

FIGS. 13A to 13C show examples of graphs of sensitivity, specificity, and precision of various techniques used to classify a result as signal or noise at different titrations in accordance with some embodiments of the disclosed subject matter. The graphs in FIGS. 13A to 13C show comparisons of performance of NN models NN6 to NN13. As shown in FIGS. 13A to 13C, the NN models generally outperformed the explicit threshold model at every concentration in sensitivity and precision, and outperformed the explicit threshold model at low concentrations in specificity, although NN6 performed about on par with the explicit threshold model on precision as concentration increased. The NN10 model generally exhibited consistently high sensitivity, specificity, and precision at each concentration.

### Further Examples Having a Variety of Features

Implementation examples are described in the following numbered clauses:

1. A method for classifying a genetic sequencing result for a sample, the method comprising: receiving a plurality of negative control sample genetic sequencing results corresponding to a respective plurality of negative control samples, each negative control sample genetic sequencing result comprises a plurality of values that are each indicative of a number of reads detected in the respective negative control sample for a respective organism of a plurality of organisms; generating a model based on the plurality of negative control sample genetic sequencing results; receiving a clinical sample genetic sequencing result for a clinical sample, the clinical sample genetic sequencing result comprising a plurality of values that are each indicative of a number of reads detected in the clinical sample for a respective organism of the plurality of organisms; identifying, utilizing the model, any values in the clinical sample genetic sequencing result that are likely to be diagnostically significant; generating a report based on the clinical sample genetic sequencing result and any organisms associated with a value identified as likely to be diagnostically significant; and causing at least a portion of the report to be presented to a user with an indication of any organisms associated with a value identified as likely to be diagnostically significant.

2. A method for classifying a genetic sequencing result for a sample, the method comprising: receiving a clinical sample genetic sequencing result for a clinical sample, the clinical sample genetic sequencing result comprising a plurality of values that are each indicative of a number of reads detected in the clinical sample for a respective organism of the plurality of organisms; identifying, utilizing a model, any values in the clinical sample genetic sequencing result that are likely to be diagnostically significant, wherein the model was generated based on a plurality of negative control sample genetic sequencing results; generating a report based on the clinical sample genetic sequencing result and any organisms associated with a value identified as likely to be diagnostically significant; and causing at least a portion of the report to be presented to a user with an indication of any organisms associated with a value identified as likely to be diagnostically significant.

3. The method of any one of clauses 1 or 2, further comprising: generating a distribution for each of the plurality of organisms based on the plurality of negative control sample genetic sequencing results; associating, for each of the plurality of organisms, a threshold that is based on the distribution; and identifying, utilizing the model, any values in the clinical sample genetic sequencing result that are likely to be diagnostically significant based on the threshold associated with the organism.

4. The method of clause 3, further comprising setting the threshold for each of the plurality of organisms at the median of the distribution associated with that organism.

5. The method of any one of clauses 1 to 4, further comprising: training a neural network using the plurality of negative control sample genetic sequencing results; providing the clinical sample genetic sequencing result as input to the trained neural network; and receiving, from the trained neural network, output identifying any values in the clinical sample genetic sequencing result that are likely to be diagnostically significant.

6. The method of clause 5, wherein the neural network is an autoencoder comprising: an input layer, the input layer comprising a plurality of nodes corresponding to respective organisms of the plurality of organisms; at least one encoding layer comprising no more than 30% of the number of nodes in the input layer; a coding layer comprising no more than 10% of the number of nodes in the input layer; at least one decoding layer comprising the same number of nodes as the at least one encoding layer; and an output layer, the output layer comprising a plurality of nodes corresponding to respective organisms of the plurality of organisms.

7. The method of clause 6, wherein the encoding layer comprises no more than 10% of the number of nodes in the input layer, and the coding layer comprises no more than 0.2% of the number of nodes in the input layer.

8. The method of clause 6, wherein the encoding layer comprises no more than 15% of the number of nodes in the input layer, and the coding layer comprises no more than 6.5% of the number of nodes in the input layer.

9. The method of any one of clauses 6 to 8, further comprising: receiving a plurality of positive control sample genetic sequencing results corresponding to a respective plurality of positive control samples, each positive control sample genetic sequencing result comprises a plurality of values that are each indicative of a number of reads detected in the respective positive control sample for a respective organism of a second plurality of organisms; and training the autoencoder using the plurality of negative control sample genetic sequencing results and the plurality of positive control sample genetic sequencing results.

10. The method of any one of clauses 1 to 9, further comprising: generating a heatmap indicative of the values in the clinical sample genetic sequencing result; augmenting which the organisms are presented in the heatmap based on any organisms associated with a value identified as likely to be diagnostically significant; and causing at least a portion of the heatmap corresponding to the organisms associated with a value identified as likely to be diagnostically significant to be presented within the portion of the report.

11. A method for classifying a genetic sequencing result for a sample, the method comprising: receiving a plurality of negative control sample genetic sequencing results corresponding to a respective plurality of negative control samples, each negative control sample genetic sequencing result comprises a plurality of values that are each indicative of a number of reads detected in the respective negative control sample for a respective group of organisms of a plurality of groups of organisms; generating a model based on the plurality of negative control sample genetic sequencing results; receiving a clinical sample genetic sequencing result for a clinical sample, the clinical sample genetic sequencing result comprising a plurality of values that are each indicative of a number of reads detected in the clinical sample for a respective group of organisms of the plurality of groups of organisms; identifying, utilizing the model, any values in the clinical sample genetic sequencing result that are likely to be diagnostically significant; generating a report based on the clinical sample genetic sequencing result and any groups of organisms associated with a value identified as likely to be diagnostically significant; and causing at least a portion of the report to be presented to a user with an indication of any groups of organisms associated with a value identified as likely to be diagnostically significant.

12. A method for classifying a genetic sequencing result for a sample, the method comprising: receiving a clinical sample genetic sequencing result for a clinical sample, the clinical sample genetic sequencing result comprising a plurality of values that are each indicative of a number of reads detected in the clinical sample for a respective group of organisms of the plurality of groups of organisms; identifying, utilizing a model, any values in the clinical sample genetic sequencing result that are likely to be diagnostically significant, wherein the model was generated based on a plurality of negative control sample genetic sequencing results; generating a report based on the clinical sample genetic sequencing result and any groups of organisms associated with a value identified as likely to be diagnostically significant; and causing at least a portion of the report to be presented to a user with an indication of any groups of organisms associated with a value identified as likely to be diagnostically significant.

13. The method of any one of clauses 11 or 12, further comprising: generating a distribution for each of the plurality of groups of organisms based on the plurality of negative control sample genetic sequencing results; associating, for each of the plurality of groups organisms, a threshold that is based on the distribution; and identifying, utilizing the model, any values in the clinical sample genetic sequencing result that are likely to be diagnostically significant based on the threshold associated with the group of organisms.

14. The method of clause 13, further comprising setting the threshold for each of the plurality of groups of organisms at the median of the distribution associated with that group of organisms.

15. The method of any one of clauses 11 to 14, further comprising: training a neural network using the plurality of negative control sample genetic sequencing results; providing the clinical sample genetic sequencing result as input to the trained neural network; and receiving, from the trained neural network, output identifying any values in the clinical sample genetic sequencing result that are likely to be diagnostically significant.

16. The method of clause 15, wherein the neural network is an autoencoder comprising: an input layer, the input layer comprising a plurality of nodes corresponding to respective group of organisms of the plurality of groups of organisms; at least one encoding layer comprising no more than 30% of the number of nodes in the input layer; a coding layer comprising no more than 10% of the number of nodes in the input layer; at least one decoding layer comprising the same number of nodes as the at least one encoding layer; and an output layer, the output layer comprising a plurality of nodes corresponding to respective group of organisms of the plurality of groups of organisms.

17. The method of clause 16, wherein the encoding layer comprises no more than 10% of the number of nodes in the input layer, and the coding layer comprises no more than 0.2% of the number of nodes in the input layer.

18. The method of clause 16, wherein the encoding layer comprises no more than 15% of the number of nodes in the input layer, and the coding layer comprises no more than 6.5% of the number of nodes in the input layer.

19. The method of any one of clauses 16 to 18, further comprising: receiving a plurality of positive control sample genetic sequencing results corresponding to a respective plurality of positive control samples, each positive control sample genetic sequencing result comprises a plurality of values that are each indicative of a number of reads detected in the respective positive control sample for a respective group of organisms of a second plurality of groups of organisms; and training the autoencoder using the plurality of negative control sample genetic sequencing results and the plurality of positive control sample genetic sequencing results.

20. The method of any one of clauses 11 to 19, further comprising: generating a heatmap indicative of the values in the clinical sample genetic sequencing result; augmenting which of the groups of organisms are presented in the heatmap based on any groups of organisms associated with a value identified as likely to be diagnostically significant; and causing at least a portion of the heatmap corresponding to the groups of organisms associated with a value identified as likely to be diagnostically significant to be presented within the portion of the report.

21. The method of any one of clauses 11 to 20, wherein the plurality of groups of organisms includes at least one group that includes multiple subspecies associated with a species.

22. The method of any one of clauses 11 to 21, wherein the plurality of groups of organisms includes at least one group that includes multiple species associated with a genus.

23. A method for classifying a genetic sequencing result for a sample, the method comprising: receiving a plurality of negative control sample genetic sequencing results corresponding to a respective plurality of negative control samples, each negative control sample genetic sequencing result comprises a plurality of values that are each indicative of a number of reads detected in the respective negative control sample for a respective taxon of a plurality of taxons; generating a model based on the plurality of negative control sample genetic sequencing results; receiving a clinical sample genetic sequencing result for a clinical sample, the clinical sample genetic sequencing result comprising a plurality of values that are each indicative of a number of reads detected in the clinical sample for a respective taxon of the plurality of taxons; identifying, utilizing the model, any values in the clinical sample genetic sequencing result that are likely to be diagnostically significant; generating a report based on the clinical sample genetic sequencing result and any taxons associated with a value identified as likely to be diagnostically significant; and causing at least a portion of the report to be presented to a user with an indication of any taxons associated with a value identified as likely to be diagnostically significant.

24. A method for classifying a genetic sequencing result for a sample, the method comprising: receiving a clinical sample genetic sequencing result for a clinical sample, the clinical sample genetic sequencing result comprising a plurality of values that are each indicative of a number of reads detected in the clinical sample for a respective taxon of the plurality of taxons; identifying, utilizing the model, any values in the clinical sample genetic sequencing result that are likely to be diagnostically significant, wherein the model was generated based on a plurality of negative control sample genetic sequencing results; generating a report based on the clinical sample genetic sequencing result and any taxons associated with a value identified as likely to be diagnostically significant; and causing at least a portion of the report to be presented to a user with an indication of any taxons associated with a value identified as likely to be diagnostically significant.

25. The method of any one of clauses 23 or 24, wherein the plurality of taxons includes taxons of different ranks.

26. The method of any one of clauses 23 to 26, further comprising: generating a distribution for each of the plurality of taxons based on the plurality of negative control sample genetic sequencing results; associating, for each of the plurality of taxons, a threshold that is based on the distribution; and identifying, utilizing the model, any values in the clinical sample genetic sequencing result that are likely to be diagnostically significant based on the threshold associated with the taxon.

27. The method of clause 26, further comprising setting the threshold for each of the plurality of taxons at the median of the distribution associated with that taxon.

28. The method of any one of clauses 23 to 27, further comprising: training a neural network using the plurality of negative control sample genetic sequencing results; providing the clinical sample genetic sequencing result as input to the trained neural network; and receiving, from the trained neural network, output identifying any values in the clinical sample genetic sequencing result that are likely to be diagnostically significant.

29. The method of clause 28, wherein the neural network is an autoencoder comprising: an input layer, the input layer comprising a plurality of nodes corresponding to respective taxons of the plurality of taxons; at least one encoding layer comprising no more than 30% of the number of nodes in the input layer; a coding layer comprising no more than 10% of the number of nodes in the input layer; at least one decoding layer comprising the same number of nodes as the at least one encoding layer; and an output layer, the output layer comprising a plurality of nodes corresponding to respective taxons of the plurality of taxons.

30. The method of clause 28, wherein the encoding layer comprises no more than 10% of the number of nodes in the input layer, and the coding layer comprises no more than 0.2% of the number of nodes in the input layer.

31. The method of clause 28, wherein the encoding layer comprises no more than 15% of the number of nodes in the input layer, and the coding layer comprises no more than 6.5% of the number of nodes in the input layer.

32. The method of any one of clauses 29 to 31, further comprising: receiving a plurality of positive control sample genetic sequencing results corresponding to a respective plurality of positive control samples, each positive control sample genetic sequencing result comprises a plurality of values that are each indicative of a number of reads detected in the respective positive control sample for a respective taxon of a second plurality of taxons; and training the autoencoder using the plurality of negative control sample genetic sequencing results and the plurality of positive control sample genetic sequencing results.

33. The method of any one of clauses 23 to 32, further comprising: generating a heatmap indicative of the values in the clinical sample genetic sequencing result; augmenting which the taxons are presented in the heatmap based on any taxons associated with a value identified as likely to be diagnostically significant; and causing at least a portion of the heatmap corresponding to the taxons associated with a value identified as likely to be diagnostically significant to be presented within the portion of the report.

34. A system comprising: at least one hardware processor that is programmed to: perform a method of any of clauses 1 to 33.

35. A non-transitory computer readable medium containing computer executable instructions that, when executed by a processor, cause the processor to perform a method of any of clauses 1 to 33.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

It should be noted that, as used herein, the term mechanism can encompass hardware, software, firmware, or any suitable combination thereof.

It should be understood that the above described steps of the processes of FIGS. 3 to 5 can be executed or performed in any order or sequence not limited to the order and sequence shown and described in the figures. Also, some of the above steps of the processes of FIGS. 3 to 5 can be executed or performed substantially simultaneously where appropriate or in parallel to reduce latency and processing times.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

What is claimed is:

1. A system for classifying a genetic sequencing result for a sample, the system comprising:
at least one hardware processor that is programmed to:
receive a plurality of negative control sample genetic sequencing results corresponding to a respective plurality of negative control samples,
each negative control sample genetic sequencing result comprises a plurality of values that are each indicative of a number of reads detected in the respective negative control sample for a respective organism of a plurality of organisms;
generate a model based on the plurality of negative control sample genetic sequencing results;
receive a clinical sample genetic sequencing result for a clinical sample,
the clinical sample genetic sequencing result comprising a plurality of values that are each indicative of a number of reads detected in the clinical sample for a respective organism of the plurality of organisms;
identify, utilizing the model, any values in the clinical sample genetic sequencing result that are likely to be diagnostically significant;
generate a report based on the clinical sample genetic sequencing result and any organisms associated with a value identified as likely to be diagnostically significant; and
cause at least a portion of the report to be presented to a user with an indication of any organisms associated with a value identified as likely to be diagnostically significant.

2. The system of claim 1, wherein the at least one hardware processor is further programmed to:
generate a distribution for each of the plurality of organisms based on the plurality of negative control sample genetic sequencing results;
associate, for each of the plurality of organisms, a threshold that is based on the distribution; and
identify, utilizing the model, any values in the clinical sample genetic sequencing result that are likely to be diagnostically significant based on the threshold associated with the organism.

3. The system of claim 2, wherein the at least one hardware processor is further programmed to set the threshold for each of the plurality of organisms at the median of the distribution associated with that organism.

4. The system of claim 1, wherein the at least one hardware processor is further programmed to:
train a neural network using the plurality of negative control sample genetic sequencing results;
provide the clinical sample genetic sequencing result as input to the trained neural network; and
receive, from the trained neural network, output identifying any values in the clinical sample genetic sequencing result that are likely to be diagnostically significant.

5. The system of claim 4, wherein the neural network is an autoencoder comprising:
an input layer, the input layer comprising a plurality of nodes corresponding to respective organisms of the plurality of organisms;
at least one encoding layer comprising no more than 15% of the number of nodes in the input layer;
a coding layer comprising no more than 6.5% of the number of nodes in the input layer;
at least one decoding layer comprising the same number of nodes as the at least one encoding layer; and
an output layer, the output layer comprising a plurality of nodes corresponding to respective organisms of the plurality of organisms.

6. The system of claim 5, wherein the encoding layer comprises no more than 10% of the number of nodes in the input layer, and the coding layer comprises no more than 0.2% of the number of nodes in the input layer.

7. The system of claim 5, wherein the at least one hardware processor is further programmed to:
receive a plurality of positive control sample genetic sequencing results corresponding to a respective plurality of positive control samples,
each positive control sample genetic sequencing result comprises a plurality of values that are each indicative of a number of reads detected in the respective positive control sample for a respective organism of a second plurality of organisms; and
train the autoencoder using the plurality of negative control sample genetic sequencing results and the plurality of positive control sample genetic sequencing results.

8. The system of claim 1, wherein the at least one hardware processor is further programmed to:
generate a heatmap indicative of the values in the clinical sample genetic sequencing result;
augment which the organisms are presented in the heatmap based on any organisms associated with a value identified as likely to be diagnostically significant; and
cause at least a portion of the heatmap corresponding to the organisms associated with a value identified as likely to be diagnostically significant to be presented within the portion of the report.

9. A method for classifying a genetic sequencing result for a sample, the method comprising:
receiving a plurality of negative control sample genetic sequencing results corresponding to a respective plurality of negative control samples,
each negative control sample genetic sequencing result comprises a plurality of values that are each indicative of a number of reads detected in the respective negative control sample for a respective organism of a plurality of organisms;
generating a model based on the plurality of negative control sample genetic sequencing results;
receiving a clinical sample genetic sequencing result for a clinical sample,
the clinical sample genetic sequencing result comprising a plurality of values that are each indicative of a number of reads detected in the clinical sample for a respective organism of the plurality of organisms;
identifying, utilizing the model, any values in the clinical sample genetic sequencing result that are likely to be diagnostically significant;
generating a report based on the clinical sample genetic sequencing result and any organisms associated with a value identified as likely to be diagnostically significant; and
causing at least a portion of the report to be presented to a user with an indication of any organisms associated with a value identified as likely to be diagnostically significant.

10. The method of claim 9, further comprising:
generating a distribution for each of the plurality of organisms based on the plurality of negative control sample genetic sequencing results;
associating, for each of the plurality of organisms, a threshold that is based on the distribution; and
identifying, utilizing the model, any values in the clinical sample genetic sequencing result that are likely to be diagnostically significant based on the threshold associated with the organism.

11. The method of claim 10, further comprising setting the threshold for each of the plurality of organisms at the median of the distribution associated with that organism.

12. The method of claim 9, further comprising:
training a neural network using the plurality of negative control sample genetic sequencing results;
providing the clinical sample genetic sequencing result as input to the trained neural network; and
receiving, from the trained neural network, output identifying any values in the clinical sample genetic sequencing result that are likely to be diagnostically significant.

13. The method of claim 12, wherein the neural network is an autoencoder comprising:
an input layer, the input layer comprising a plurality of nodes corresponding to respective organisms of the plurality of organisms;
at least one encoding layer comprising no more than 15% of the number of nodes in the input layer;
a coding layer comprising no more than 6.5% of the number of nodes in the input layer;
at least one decoding layer comprising the same number of nodes as the at least one encoding layer; and
an output layer, the output layer comprising a plurality of nodes corresponding to respective organisms of the plurality of organisms.

14. The method of claim 13, further comprising:
receiving a plurality of positive control sample genetic sequencing results corresponding to a respective plurality of positive control samples,
each positive control sample genetic sequencing result comprises a plurality of values that are each indicative of a number of reads detected in the respective positive control sample for a respective organism of a second plurality of organisms; and
training the autoencoder using the plurality of negative control sample genetic sequencing results and the plurality of positive control sample genetic sequencing results.

15-22. (canceled)

23. The method of claim 9, further comprising:
generating a heatmap indicative of the values in the clinical sample genetic sequencing result;
augmenting which the organisms are presented in the heatmap based on any organisms associated with a value identified as likely to be diagnostically significant; and
causing at least a portion of the heatmap corresponding to the organisms associated with a value identified as likely to be diagnostically significant to be presented within the portion of the report.

24. A non-transitory computer readable medium containing computer executable instructions that, when executed by a processor, cause the processor to perform a method for classifying a genetic sequencing result for a sample, the method comprising:
receiving a plurality of negative control sample genetic sequencing results corresponding to a respective plurality of negative control samples,
each negative control sample genetic sequencing result comprises a plurality of values that are each indicative of a number of reads detected in the respective negative control sample for a respective organism of a plurality of organisms;
generating a model based on the plurality of negative control sample genetic sequencing results;
receiving a clinical sample genetic sequencing result for a clinical sample,
the clinical sample genetic sequencing result comprising a plurality of values that are each indicative of a number of reads detected in the clinical sample for a respective organism of the plurality of organisms;
identifying, utilizing the model, any values in the clinical sample genetic sequencing result that are likely to be diagnostically significant;
generating a report based on the clinical sample genetic sequencing result and any organisms associated with a value identified as likely to be diagnostically significant; and
causing at least a portion of the report to be presented to a user with an indication of any organisms associated with a value identified as likely to be diagnostically significant.

25. The non-transitory computer readable medium of claim 24, wherein the method further comprises:
generating a distribution for each of the plurality of organisms based on the plurality of negative control sample genetic sequencing results;
associating, for each of the plurality of organisms, a threshold that is based on the distribution; and
identifying, utilizing the model, any values in the clinical sample genetic sequencing result that are likely to be diagnostically significant based on the threshold associated with the organism.

26. The non-transitory computer readable medium of claim 25, wherein the method further comprises setting the threshold for each of the plurality of organisms at the median of the distribution associated with that organism.

27. The non-transitory computer readable medium of claim 24, wherein the method further comprises:
training a neural network using the plurality of negative control sample genetic sequencing results;
providing the clinical sample genetic sequencing result as input to the trained neural network; and
receiving, from the trained neural network, output identifying any values in the clinical sample genetic sequencing result that are likely to be diagnostically significant.

28. The non-transitory computer readable medium of claim 27, wherein the neural network is an autoencoder comprising:
an input layer, the input layer comprising a plurality of nodes corresponding to respective organisms of the plurality of organisms;
at least one encoding layer comprising no more than 15% of the number of nodes in the input layer;
a coding layer comprising no more than 6.5% of the number of nodes in the input layer;
at least one decoding layer comprising the same number of nodes as the at least one encoding layer; and
an output layer, the output layer comprising a plurality of nodes corresponding to respective organisms of the plurality of organisms.

29. The non-transitory computer readable medium of claim 28, wherein the method further comprises:
receiving a plurality of positive control sample genetic sequencing results corresponding to a respective plurality of positive control samples,
each positive control sample genetic sequencing result comprises a plurality of values that are each indicative of a number of reads detected in the respective positive control sample for a respective organism of a second plurality of organisms; and
training the autoencoder using the plurality of negative control sample genetic sequencing results and the plurality of positive control sample genetic sequencing results.

30. The non-transitory computer readable medium of claim 24, wherein the method further comprises:
generating a heatmap indicative of the values in the clinical sample genetic sequencing result;
augmenting which the organisms are presented in the heatmap based on any organisms associated with a value identified as likely to be diagnostically significant; and
causing at least a portion of the heatmap corresponding to the organisms associated with a value identified as likely to be diagnostically significant to be presented within the portion of the report.
